SEQUENCE LISTING (5 sequences)

SEQ ID NO: 1 - protein, 104 aa, Rana pipiens
EDWLTFQKKHITNTRDVDCDNIMSTNLFHCKDKNTFIYSRPEPVKAICKGIIASKNVLTTSEFYLSDCNVTSRPCKYKLKKSTNKFCVTCENQAPVHFVGVGSC

SEQ ID NO: 2 - protein, 104 aa, Artificial Sequence
  ′805 variant of ranpirnase
EDWLTFQKKHVTNTRDVDCNNIMSTNLFHCKDKNTFIYSRPEPVKAICKGIIASKNVLTTSEFYLSDCNVTSRPCKYKLKKSTNKFCVTCENQAPVHFVGVGRC

SEQ ID NO: 3 - protein, 114 aa, Rana pipiens
KPKEDREWEKFKTKHITSQSVADFNCNRTMNDPAYTPDGQCKPVNTFIHSTTGPVKEICRRATGRVNKSSTQQFTLTTCKNPIRCKYSQSNTTNFICITCRDNYPVHFVKTGKC

SEQ ID NO: 4 - protein, 114 aa, Artificial Sequence
  rAmphirnase 2
KPKEDREWEKFKTKHITSQSVADFNCNRTMNDPAYTPDGQCKPINTFIHSTTGPVKEICRRATGRVNKSSTQQFTLTTCKNPIRCKYSQSNTTNFICITCRDNYPVHFVKTGKC

SEQ ID NO: 5 - protein, 104 aa, Rana pipiens
EDWLTFQKKHITNTRDVDCDNIMSSNLFHCKDKNTFIYSRPEPVKAICKGIIASKNVLTTSEFYLSDCNVTSRPCKYKLKKSTNKFCVTCENQAPVHFVGVGSC

(19) United States
(12) Patent Application Publication
(10) Pub. No.: US 2023/0147602 A1
(43) Pub. Date: May 11, 2023

(54) RIBONUCLEASES FOR TREATING VIRAL INFECTIONS

(71) Applicant: Orgenesis Inc., Germantown, MD (US)

(72) Inventors: JAMIE SULLEY, La Jolla, CA (US); LUIS SQUIQUERA, Buenos Aires (AR); THOMAS HODGE, Athens, GA (US); SABINA GLOZMAN, Naharya (IL); Vered CAPLAN, Coppet (CH)

(73) Assignee: Orgenesis Inc., Germantown, MD (US)

(21) Appl. No.: 17/906,633

(22) PCT Filed: Mar. 19, 2021
(86) PCT No.: PCT/US2021/023094

§ 371 Date: Sep. 19, 2022

Related U.S. Application Data
(60) Provisional application No. 62/992,408, filed on Mar. 20, 2020.
(60) Provisional application No. 63/006,728, filed on Apr. 8, 2020.
(60) Provisional application No. 63/009,881, filed on Apr. 14, 2020.
(60) Provisional application No. 63/017,669, filed on Apr. 30, 2020.

(51) Int. Cl. A61K 38/46; A61K 9/107; A61P 31/14

(52) U.S. Cl. CPC A61K 38/465; A61K 9/1075; A61P 31/14

(57) ABSTRACT

This disclosure is directed to compounds and pharmaceutical compositions for treating and preventing viral diseases, as Covid-19. Among others, the invention relates to the use of ribonucleases and bioxoms, exosomes or combination thereof in the preparations and use of pharmaceutical formulations for the treatment of said disease. In addition, the invention relates to the use of immune cells and ribonucleases in the preparation and use of pharmaceutical formulations for the treatment of said disease.

### FIELD OF THE DISCLOSURE

This disclosure is directed to compounds and pharmaceutical compositions for treating and preventing viral diseases, as Covid-19. Among others, the invention relates to the use of ribonucleases and bioxoms, exosomes or combination thereof in the preparations and use of pharmaceutical formulations for the treatment of said disease. In addition, the invention relates to the use of immune cells and ribonucleases in the preparation and use of pharmaceutical formulations for the treatment of said disease.

### SEQUENCE LISTING STATEMENT

The instant application contains a Sequence Listing which has been submitted electronically in ASCII format and is hereby incorporated by reference in its entirety. Said ASCII copy, created on Mar. 19, 2021, is named P-603949-PC-SQL_ST25.txt and is 5,329 bytes in size.

### BACKGROUND

Several human diseases are caused by viruses, as the common cold, influenza, chickenpox, cold sores, rabies, Ebola virus disease, AIDS (HIV), avian influenza, SARS, and Covid-19. These diseases are usually detected by clinical presentation, for instance severe muscle and joint pains preceding fever, or skin rash and swollen lymph glands.

Coronaviruses (CoV) are a large family of viruses that cause illness ranging from the common cold to more severe diseases such as Middle East Respiratory Syndrome (MERS-CoV), Severe Acute Respiratory Syndrome (SARS-CoV), and Covid-19. Coronaviruses are in the subfamily Orthocoronavirinae in the family Coronaviridae, in the order Nidovirales. They are enveloped viruses with a positive-sense single-stranded RNA genome and a nucleocapsid of helical symmetry. The genome size of coronaviruses ranges from approximately 26 to 32 kilobases, the largest for an RNA virus. Coronaviruses are zoonotic, meaning they are transmitted between animals and people. Coronaviruses further cause colds with major symptoms, such as fever and sore throat from swollen adenoids, primarily in the winter and early spring seasons, pneumonia, and bronchitis, among others.

The novel coronavirus SARS-CoV-2, informally known as the Wuhan coronavirus, is a contagious virus that causes acute respiratory diseases, and has been the cause of a major virus outbreak known as 2019-20 Wuhan coronavirus outbreak. The virus is thought to have a zoonotic origin, as suggested by its similarity to SARS-CoV and bat coronaviruses. However, human-to-human transmission of the virus has been confirmed, primarily through close contact, in particular through respiratory droplets from coughs and sneezes. Viral RNA has also been found in stool samples from infected patients.

There are no antiviral drugs to prevent or treat human coronavirus infections.

### SUMMARY OF THE INVENTION

In one aspect, disclosed herein is a composition comprising a ribonuclease and a bioxome, an exosome or a combination thereof. In some related aspects, the ribonuclease is selected from a group comprising RNase A, RNase H, RNase III, RNase L, RNase P, RNase PhyM, RNase T1, RNase T2, RNase U2, RNase V, PNPase, RNase PH, RNase R, RNase D, RNase T, oligoribonuclease, exoribonuclease I, exoribonuclease II, binase, MCPIP1, eosinophil cationic protein (ECP), eosinophil derived neurotoxin (EDN), RNase 3, ranpirnase, rAmphinase, rAmphinase 2, bovine seminal RNase (BS_RNase).

In some related aspects, the ribonuclease comprises ranpirnase. In some related aspects the composition is for use in treating a viral disease. In some related aspects, the viral disease is caused by a virus selected from a group comprising severe acute respiratory syndrome coronavirus 2 (SARS-CoV-2), an adenovirus, a herpesvirus, a papillomavirus, a polyomavirus, a poxvirus, an hepadnavirus, a parvovirus, an astrovirus, a calicivirus, a picornavirus, a coronavirus, a flavivirus, a togavirus, a hepevirus, a retrovirus, an orthomyxovirus, an arenavirus, a bunyavirus, a filovirus, a paramyxovirus, a rhabdovirus, a reovirus, Herpes simplex type 1, Herpes simplex type 2, Varicella-zoster virus, Epstein-Barr virus, Human cytomegalovirus, human herpesvirus type 8, human papillomavirus, BK virus, JC virus, smallpox, Hepatitis B virus, parvovirus B19, human astrovirus, Norwalk virus, coxsackievirus, hepatitis A virus, poliovirus, rhinovirus, severe acute respiratory syndrome virus, hepatitis C virus, yellow fever virus, dengue virus, West Nile virus, TBE virus, Rubella virus, Hepatitis E virus, Human immunodeficiency virus (HIV), Influenza virus, Lassa virus, Crimean-Congo hemorrhagic fever virus, Hantaan virus, Ebola virus, Marburg virus, Measles virus, Mumps virus, Parainfluenza virus, Respiratory syncytial virus, Rabies virus, Hepatitis D, Rotavirus, Orbivirus, Coltivirus, Banna virus, or any combination thereof.

In some related aspects, the viral disease is selected from a group comprising acute hepatitis, AIDS, aseptic meningitis, bronchiolitis, Burkitt's lymphoma, chickenpox, chronic hepatitis, common cold, congenital rubella, congenital varicella syndrome, congenital seizures in the newborn, croup, cystitis, cytomegalic inclusion disease, fatal encephalitis, gastroenteritis, German measles, gingivostomatitis, hepatic cirrhosis, hepatocellular carcinoma, herpes labialis, cold sores, herpes zoster, Hodgkin's lymphoma, hyperplastic epithelial lesions, warts, laryngeal papillomas, epidermodysplasia verruciformis, infectious mononucleosis, influenza, influenza-like syndrome, Kaposi sarcoma, keratoconjunctivitis, liver, lung and spleen diseases in the newborn, malignancies, cervical carcinoma, squamous cell carcinomas, measles, multicentric Castleman disease, mumps, myocarditis, nasopharyngeal carcinoma, pericarditis, pharyngitis, pharyngoconjunctival fever, pleurodynia, pneumonia, poliomyelitis, postinfectious encephalomyelitis, premature delivery, primary effusion lymphoma, rabies, Reye syndrome, severe bronchiolitis with pneumonia, skin vesicles, mucosal ulcers, tonsillitis, pharyngitis, or combination thereof.

In some related aspects, the viral disease comprises Covid-19, or wherein said viral disease is caused by severe acute respiratory syndrome coronavirus 2 (SARS-CoV-2). In some related aspects, the composition further comprises immunoglobulins, fragments thereof, antibodies, or combinations thereof, obtained from a plasma of a subject immune to said viral disease.

In some related aspects, the immunoglobulins are IgG, IgM or combinations thereof. In some related aspects, the immunoglobulin fragments are F(ab′)2 fragments. In some related aspects, the viral disease comprises Covid-19, and said plasma is collected from healthy subject who have been previously exposed to SARS-CoV-2, naturally or by deliberate immunization, and who have IgG or IgM antibodies to SARS-CoV-2 virus in their plasma.

In some related aspects, the viral disease comprises Covid-19, and said plasma is collected from a subject or pool of subjects where SARS-CoV-2 infection rate is high. In some related aspects, the viral disease comprises Covid-19, and said plasma is collected from a subject or pool of subjects who have a history of SARS-CoV-2 infection in the past. In some related aspects, the viral disease comprises Covid-19, and said plasma is collected from a subject or pool of subjects who are found to have IgG or IgM antibodies to SARS-CoV-2 through an antibody screening program.

In some related aspects, the viral disease comprises Covid-19, and said plasma is collected from a subject or pool of subjects who have antibodies as the result of deliberate immunization with SARS-CoV-2 or with antigens associated with SARS-CoV-2. In some related aspects, the viral disease comprises Covid-19, and said plasma is collected by either plasmapheresis or after separation from whole blood donations.

In some related aspects, the composition further comprises immune cells. In some related aspects, the immune cells are selected from a group comprising neutrophils, eosinophils (acidophiles), basophils, lymphocytes, monocytes, B cells, memory B cell, regulatory B cells (Breg), T cells, cytotoxic T cells, Helper T cells, Th1 cells, Th2 cells, Regulatory T cells (Treg), memory T cells, Natural Killer (NK) cells, monocytes, dendritic cells, macrophages, myeloid dendritic cells (mDC), plasmacytoid dendritic cell (pDC), or a combination thereof.

In some related aspects, the immune cells comprise NK cells. In some related aspects, the immune cells are obtained from a donor, or from a cell line. In some related aspects, the immune cells are obtained from a subject immune to said viral disease. In some related aspects, the composition is delivered by intranasal route using an aerosol spray.

In some aspects, disclosed herein is a composition comprising a ribonuclease and immunoglobulins, fragments thereof, antibodies, or combinations thereof, obtained from a plasma of a subject immune to a viral disease.

In one aspect, disclosed herein is a method for treating a viral disease in a subject in need thereof, the method comprising administering a composition comprising a ribonuclease and a bioxome, an exosome or a combination thereof.

In one aspect, disclosed herein is a composition for treating or preventing a viral disease in a subject, said composition comprising a ribonuclease. In some related aspects, the composition is loaded into a bioxome, an exosome, or a combination thereof. In some related aspects, the composition is administered with a bioxome, an exosome, or a combination thereof. In some aspects, disclosed herein is a composition comprising a ribonuclease and immune cells.

### BRIEF DESCRIPTION OF THE DRAWINGS

The subject matter disclosed herein is particularly pointed out and distinctly claimed in the concluding portion of the specification. The compositions and methods for using thereof disclosed herein may best be understood by reference to the following detailed description when read with the accompanying drawings in which:

FIG. 1 shows an assay of ranpirnase and FDA-Approved Drugs efficacy against SARS-CoV-2 in vitro.

### DETAILED DESCRIPTION

In some embodiments, disclosed herein is a composition comprising a ribonuclease and a bioxome, an exosome or a combination thereof. In one embodiment, disclosed herein is a composition comprising ranpirnase and a bioxome. In another embodiment, disclosed herein is a composition comprising ranpirnase and an exosome. In another embodiment, disclosed herein is a composition comprising amphinase and a bioxome. In another embodiment, disclosed herein is a composition comprising amphinase and an exosome.

### Ribonucleases

A skilled artisan would appreciate that ribonucleases, or RNases, are a type of nuclease that catalyzes the degradation of RNA into smaller components. RNases comprise a first defense against RNA viruses, and provide the underlying machinery for more advanced cellular immune strategies such as RNAi. Several types of RNases can be used to degrade RNA, all of them can be used for the compositions and methods disclosed herein.

In some embodiments, a ribonuclease comprises RNase A. In some embodiments, a ribonuclease comprises RNase H. In some embodiments, a ribonuclease comprises RNase III. In some embodiments, a ribonuclease comprises RNase L. In some embodiments, a ribonuclease comprises RNase P. In some embodiments, a ribonuclease comprises RNase PhyM. In some embodiments, a ribonuclease comprises RNase T1.

In some embodiments, a ribonuclease comprises RNase T2. In some embodiments, a ribonuclease comprises RNase U2. In some embodiments, a ribonuclease comprises RNase V. In some embodiments, a ribonuclease comprises PNPase. In some embodiments, a ribonuclease comprises RNase PH. In some embodiments, a ribonuclease comprises RNase R. In some embodiments, a ribonuclease comprises RNase D. In some embodiments, a ribonuclease comprises RNase T.

In some embodiments, a ribonuclease comprises oligoribonuclease. In some embodiments, a ribonuclease comprises exoribonuclease I. In some embodiments, a ribonuclease comprises exoribonuclease II. In some embodiments, a ribonuclease comprises binase. In some embodiments, a ribonuclease comprises MCPIP1. In some embodiments, a ribonuclease comprises eosinophil cationic protein (ECP). In some embodiments, a ribonuclease comprises eosinophil derived neurotoxin (EDN).

In some embodiments, a ribonuclease comprises RNase 3. In some embodiments, a ribonuclease comprises onconase. In some embodiments, a ribonuclease comprises rAmphinase. In some embodiments, a ribonuclease comprises rAmphinase 2. In some embodiments, a ribonuclease comprises bovine seminal RNase (BS_RNase).

In some embodiments, a ribonuclease comprises a human ribonuclease. In some embodiments, a ribonuclease comprises a mammalian ribonuclease. In some embodiments, a comprises a microbial ribonuclease. In some embodiments, a ribonuclease comprises a frog ribonuclease. In some embodiments, a ribonuclease comprises a frog oocytes ribonuclease. In some embodiments, a ribonuclease comprises an artificial ribonuclease. In some embodiments, more than one type of ribonuclease is used in the compositions and methods disclosed herein.

In some embodiments, a ribonuclease degrades tRNA. In some embodiments, a ribonuclease degrades rRNA. In some embodiments, a ribonuclease degrades mRNA. In some embodiments, a ribonuclease is conjugated to a molecule. In some embodiments, a ribonuclease is conjugated to human serum albumin.

In some embodiments, a ribonuclease comprises ranpirnase. In some embodiments, disclosed herein is a composition comprising ranpirnase and immunoglobulins, fragments thereof, antibodies, or combinations thereof, obtained from a plasma of a subject immune to a viral disease. In some embodiments, disclosed herein is a composition comprising ranpirnase and immune cells. In some embodiments, disclosed herein is a composition comprising ranpirnase, immunoglobulins, fragments thereof, antibodies, or combinations thereof, obtained from a plasma of a subject immune to said viral disease, and immune cells. In some embodiments, disclosed herein is a composition comprising ranpirnase, immunoglobulins, fragments thereof, antibodies, or combinations thereof, obtained from a plasma of a subject immune to said viral disease, and natural killer cells.

A skilled artisan would appreciate that Ranpirnase, called herein also “onconase”, “P-30”, “TMR004”, and “Pannon”, is a ribonuclease enzyme found in the oocytes of the Northern Leopard Frog (Rana pipiens). Ranpirnase is a member of the pancreatic ribonuclease (RNase A) protein superfamily and degrades RNA substrates with a sequence preference for uracil and guanine nucleotides. Ranpirnase has been studied as a potential cancer and antiviral treatment due to its unusual mechanism of cytotoxicity tested against transformed cells and antiviral activity. Ranpirnase UniProt identification number is P85073.

In some embodiments, ranpirnase comprises an amino acid sequence comprising EDWLTFQKKHITNTRDVDCDNIMSTNLFHCKDKNTFIYSRPEPVKAICKGIIASKN VLTTSEFYLSDCNVTSRPCKYKLKKSTNKFCVTCENQAPVHFVGVGSC (SEQ ID No.: 1). In some embodiments, ranpirnase comprises an amino acid sequence comprising at least 80%, 85%, 90%, 95%, or 99% homology to SEQ ID No.:1.

In some embodiments, ranpirnase comprises an amino acid sequence comprising EDWLTFQKKHVTNTRDVDCNNIMSTNLFHCKDKNTFIYSRPEPVKAICKGIIASK NVLTTSEFYLSDCNVTSRPCKYKLKKSTNKFCVTCENQAPVHFVGVGRC (SEQ ID No.: 2). In some embodiments, ranpirnase comprises an amino acid sequence comprising at least 80%, 85%, 90%, 95%, or 99% homology to SEQ ID No.:2.

In some embodiments, ranpirnase comprises an amino acid sequence comprising EDWLTFQKKHITNTRDVDCDNIMSSNLFHCKDKNTFIYSRPEPVKAICKGIIASKN VLTTSEFYLSDCNVTSRPCKYKLKKSTNKFCVTCENQAPVHFVGVGSC (SEQ ID No.: 5). In some embodiments, ranpirnase comprises an amino acid sequence comprising at least 80%, 85%, 90%, 95%, or 99% homology to SEQ ID No.:5.

In some embodiments, a ribonuclease comprises amphinase. In some embodiments, disclosed herein is a composition comprising amphinase and immunoglobulins, fragments thereof, antibodies, or combinations thereof, obtained from a plasma of a subject immune to a viral disease. In some embodiments, disclosed herein is a composition comprising amphinase and immune cells. In some embodiments, disclosed herein is a composition comprising amphinase, immunoglobulins, fragments thereof, antibodies, or combinations thereof, obtained from a plasma of a subject immune to said viral disease, and immune cells. In some embodiments, disclosed herein is a composition comprising amphinase, immunoglobulins, fragments thereof, antibodies, or combinations thereof, obtained from a plasma of a subject immune to said viral disease, and natural killer cells.

A skilled artisan would appreciate that “amphinase”, termed herein also “amphinase 2” and “ramphinase”, is a ribonuclease enzyme found in the oocytes of the Northern leopard frog (Rana pipiens). Amphinase is a member of the pancreatic ribonuclease protein superfamily and degrades long RNA substrates, and has been studied as a potential cancer therapy due to its unusual mechanism of cytotoxicity tested against tumor cells.

In some embodiments, amphinase comprises an amino acid sequence comprising KPKEDREWEKFKTKHITSQSVADFNCNRTMNDPAYTPDGQCKPVNTFIHSTTGP VKEICRRATGRVNKSSTQQFTLTTCKNPIRCKYSQSNTTNFICITCRDNYPVHFVK TGKC (SEQ ID No.: 3). In some embodiments, amphinase comprises an amino acid sequence comprising at least 80%, 85%, 90%, 95%, or 99% homology to SEQ ID No.:3.

In some embodiments, amphinase comprises an amino acid sequence comprising KPKEDREWEKFKTKHITSQSVADFNCNRTMNDPAYTPDGQCKPINTFIHSTTGPV KEICRRATGRVNKSSTQQFTLTTCKNPIRCKYSQSNTTNFICITCRDNYPVHFVKT GKC (SEQ ID No.: 4). In some embodiments, amphinase comprises an amino acid sequence comprising at least 80%, 85%, 90%, 95%, or 99% homology to SEQ ID No.:4.

A skilled artisan would appreciate that ranpirnase and amphinase are RNAse a enzymes. RNAse III enzymes are in the RNAse C family that recognizes double stranded RNA, which the severe acute respiratory syndrome coronavirus 2 (SARS-CoV-2) is not.

A skilled artisan would appreciate that different ribonucleases exert their antiviral activity by different mechanisms. All are relevant to the compositions and methods of the present disclosure. In some embodiments, a ribonuclease enters into the cells via receptor-mediated endocytosis and once internalized into the cytosol, selectively degrades tRNA, resulting in inhibition of protein synthesis and induction of cell apoptosis.

In some embodiments, conjugation, co-encapsulation, or co-formulation of the immunoglobulins disclosed herein and a ribonuclease, results in an anti-viral immunotoxin composition. A skilled artisan will appreciate that ribonucleases are hydrophilic compounds with relatively high stability. In some embodiments, the composition is encapsulated in naturally occurring lipid membranes. In some embodiments, composition is encapsulated in nanoparticles membrane mimetics, as bioxomes.

### Bioxomes

In some embodiments, the compositions disclosed herein are loaded into artificial exosomes. In some embodiments, the ribonucleases disclosed herein are loaded into artificial exosomes. In some embodiments, the immune cells disclosed herein are loaded into artificial exosomes. In some embodiments, the immune cells disclosed herein are co-administered with artificial exosomes.

In some embodiments, the compositions disclosed herein are loaded into a naturally occurring exosomes. In some embodiments, the ribonucleases disclosed herein are loaded into a naturally occurring exosomes. In some embodiments, the immune cells disclosed herein are loaded into a naturally occurring exosomes. In some embodiments, the immune cells disclosed herein are co-administered with a naturally occurring exosomes.

In some embodiments, disclosed herein is a composition comprising a ribonuclease and an exosome, wherein the ribonuclease is loaded into the exosome. In some embodiments, disclosed herein is a composition comprising a ribonuclease and an exosome, wherein the ribonuclease is not loaded into the exosome. In some embodiments, disclosed herein is a composition comprising ribonucleases and exosomes, wherein part of the ribonucleases is loaded into the exosomes.

A skilled artisan will appreciate that exosomes are membrane bound extracellular vesicles (EVs) produced in the endosomal compartment of eukaryotic cells. In multicellular organisms, exosomes and other EVs are present in tissues and can also be found in biological fluids including blood, urine, and cerebrospinal fluid. They are also released in vitro by cultured cells into their growth medium. The multivesicular body (MVB) is an endosome defined by intraluminal vesicles (ILVs) that bud inward into the endosomal lumen. If the MVB fuses with the cell surface (the plasma membrane), these ILVs are released as exosomes. Exosomes are usually smaller than most other EVs, ranging from about 30 to 150 nanometres (nm) in diameter.

In some embodiments, an artificial exosome, which is termed herein also “bioxosome” having all the same qualities, comprises a cell membrane that undergoes fusion with a target cell and releases its cargo into that target cell after the fusion. In some embodiments, the cell membrane component is derived from a selected cellular or extracellular source. As used herein, the term “bioxome” refers, without limitation to an artificial, submicron nano-particle having resemblance to natural extracellular vesicles (EV).

In some embodiments, disclosed herein is a composition comprising a ribonuclease and a bioxome, wherein the ribonuclease is loaded into the bioxome. In some embodiments, disclosed herein is a composition comprising a ribonuclease and a bioxome, wherein the ribonuclease is not loaded into the bioxome. In some embodiments, disclosed herein is a composition comprising ribonucleases and bioxomes, wherein part of the ribonucleases is loaded into the bioxomes.

In some embodiments, the particle size of the bioxome ranges from 0.03 pm to 5 pm. In one embodiment, the size of the bioxome is 0.1-0.7 pm; 0.1-0.5 pm, 0.2-0.5 pm; 0.3-0.5 pm. In another embodiment, the average particle size is 5 pm or less; 1.5 pm or less; 0.7 pm or less; 0.5 pm or less; 0.3 pm or less; 0.15 pm or less. In one embodiment, the average particle size is 0.5 pm to 1.5 pm. In one embodiment, the average particle size is 0.4 pm to 0.8 pm. In another embodiment, the average particle size is 0.3 pm to 0.5 pm. In yet further embodiment, the average particle size is 0.4 pm to 1.5 pm when particle size is measured within few hours after the preparation. In yet another embodiment, the particle size is 0.8 pm to 5 pm when particle size is measured within a month after the preparation and bioxome particles are stored at 0° C. to −4° C.

In one embodiment, the bioxome particles are selective targeting bioxomes. In the context of the invention, the term “selective targeting bioxome” refers, without limitation, to bioxome particles designed for specific targeting ligand or homing moieties. In the selective targeting bioxome of the invention, the ligand or homing moieties are, without limitation, glycosaminoglycan; monospecific or bispecific antibodies; aptamers; receptors; fusion proteins; fusion peptides; or synthetic mimetics thereof; cancer targeting—folic acid; specific phospholipids; cytokines, growth factors; or a combination thereof.

In one embodiment, the membrane of bioxome particles of the invention comprises at least 50% from cell membrane obtained from the cellular source cultured in pre-defined cell culture conditions. In one embodiment, the bioxome particles derived from different sources may show differences in lipid composition compared to the plasma membrane.

In some embodiments, a bioxome comprises extracellular vesicles. In some embodiments, a bioxome comprises extracellular vesicles mimetics. In some embodiments, a bioxome comprises GLM. In some embodiments, a bioxome is formulated into dry submicron powder. In some embodiments, a bioxome is formulated into dry submicron powder prepared by top-down methods, such as milling, extrusion, or grinding, or by bottom up methods, such as liposomes, Bioxomes, spray dry, or freeze-dry.

In some embodiments, the cell membrane component of a bioxome is derived from a selected cellular or extracellular source by a process comprising:
a. Performing total cell lipid extraction from the selected cellular or extracellular source in a mild solvent system to obtain a lipid extract;
b. Drying the lipid extract; and c. Inducing self-assembly of bioxome particles by performing at least one step of ultra-sonication; wherein the resulting bioxome particles in the sample are characterized by an average particle size of 0.03 pm to 5 pm.
   As used herein, the term “mild solvent” refers, without limitation, to any of solvents of Class 3 or of Class 2 with PDE>2.5 mg/day and Concentration limit>250 ppm as defined by the FDA.

In one embodiment, the average particle size is 0.05 pm to 3 pm. In yet another embodiment, the average particle size is 0.08 pm to 1.5 pm. In further embodiment the average particle size is 0.1-0.7 pm; 0.1-0.5 pm, 0.2-0.5 pm; 0.3-0.5 pm. In another embodiment, the average particle size is 5 pm or less; 1.5 pm or less; 0.7 pm or less; 0.5 pm or less; 0.3 pm or less; 0.15 pm or less. In one embodiment, the average particle size is 0.5 pm to 1.5 pm. In one embodiment, the average particle size is 0.4 pm to 0.8 pm. In another embodiment, the average particle size is 0.3 pm to 0.5 pm.

In one embodiment, the sample comprising the bioxome particle has the pH of 4.5 to 5. In yet another embodiment, the sample comprising the bioxome particle has the pH of 4.5 to 5. In one embodiment, the solvent system comprises a mixture of polar and non-polar solvents. In one embodiment, the polar solvent in the solvent system is selected from the group consisting of isopropanol, ethanol, n-butanol, and water-saturated n-butanol. In one embodiment, the non-polar solvent in the solvent system is selected from hexane and solvents from the terpene group. In one embodiment, the non-polar solvent in the solvent system is n-hexane. In one embodiment, hexane may be fully or partially suspended by supercritical fluid extraction using supercritical carbon dioxide (scCO2) as a mild “green” solvent has many advantageous properties, including gas-like viscosity, liquid-like density, about 100-fold faster diffusivity than in organic solvents at ambient conditions, as well as operation at relatively low temperature. Terpene/flavonoid may be selected further from alpha-pinene, d-limonene, linalool, eucalyptol, terpineol-4-ol, p-cymene, borneol, delta-3-carene, beta-sitosterol, beta-myrcene, beta-caryophyllene, cannflavin A, apigenin, quercetin and pulegone. In one embodiment, the solvent from the terpene group is selected from the group consisting of d-limonene, a-pinene and para-cymene.

In one embodiment, the polar solvent in the solvent system is isopropanol, and the non-polar solvent is n-hexane. In yet another embodiment, the solvent is Hexane-I sopropanol 3:2 low toxicity solvent mixture. In one embodiment, the solvent system further comprises a stabilizer. In another embodiment, the stabilizer is butyl-hydroxytoluene (BHT). In one embodiment, the solvent system may further comprise additives such as, without limitation, antioxidants, surfactants stabilizers vitamin E, squalene, and cholesterol, or a combination thereof.

In one embodiment, the bioxome engineering is achieved using cell membrane collected from cellular or extracellular source through hydrophilic-hydrophobic self-assembly during cavitation ultrasonication procedure in hydrophilic vehicle. In one embodiment, bioxome particles are extruded after lipid membrane isolation post ultrasonication. In one embodiment, the cargo comprising the active molecules is hydrophilic, and is entrapped into hydrophilic vehicle during ultrasonication, or during extrusion. In yet another embodiment, the cargo is hydrophobic cargo and is entrapped prior to extraction with the solvent system, during extraction, during drying/solvent evaporation procedure, during ultrasonication, during extrusion. Repetitive freeze thawing may improve rate of encapsulation of hydrophilic cargo post drying and post ultrasonication. The level of encapsulation loading is affected by selection of engineering parameter based on sensitivity, stability and desired loading dose of selected cargo as predesigned at each specific therapeutic or research moiety.

In one embodiment, the active molecule, i.e., the ribonuclease may be interwoven into Bioxome core at predefined concentration without risk for viral gene vectors impurities as safety concerns. In one embodiment, the bioxome particles may be electroporated or microinjected. In one embodiment, RNA or DNA may be incorporated into the bioxome particles through gentle ultrasonication at 4° C. in the presence of any suitable protective buffers to maintain integrity of nucleic material for therapeutic delivery. According to the embodiments of the invention, the manufacturing process is compliant with most known industrial features of LNPs and liposomes.

In one embodiment, the cellular or extracellular source for total lipid extraction is selected from the group consisting of fibroblasts, mesenchymal stem cells, stem cells, cells of the immune system, dendritic cells, ectoderm, keratinocytes, cells of GI, cells of oral cavity, nasal mucosal cells, neuronal cells, retinal cells, endothelial cells, cardiospheres, cardiomyocytes, pericytes, blood cells, melanocytes, parenchymal cells, liver reserve cells, neural stem cells, pancreatic stem cells, embryonic stem cells, bone marrow, skin tissue, liver tissue, pancreatic tissue, postnatal umbilical cord, placenta, amniotic sac, kidney tissue, neurological tissue, biological fluids, and excrement or surgery extracted tissues, (i.e. milk, saliva, mucus, blood plasma, urine, feces, amniotic fluids, sebum, postnatal umbilical cord, placenta, amniotic sac, kidney tissue, neurological tissue, adrenal gland tissue, mucosal epithelium, smooth muscle tissue, adrenal gland tissue, mucosal epithelium, smooth muscle tissue, a bacterial cell, a bacterial culture, a whole microorganism, conditional medium, amniotic fluid, lipoaspirate, liposuction byproducts, and a plant tissue. In yet another embodiment, the lipid extraction is performed from cell-conditioned media, lyophilized conditioned cell media, cell pellet, frozen cells, dry cells, washed cell bulk, non-adhesive cell suspension, and adhesive cell layer.

In yet another embodiment, the cell layer is grown in cell culture plastic ware coated or uncoated by extracellular matrix or synthetic matrix, selected from a (multi) flask, a dish, a scaffold, beads, and a bioreactor. According to one embodiment of the invention, the membrane extract is dried by freeze or/and spray/freeze drying. In yet another embodiment, the membrane extract is dried by evaporation. The evaporation can be carried out by any suitable technique, including, but not limited to speed-vac centrifuge, argon/nitrogen blowdown, spiral air flow and other available solvent evaporation methods in controlled temperature environment, such as microwave or rotor evaporation, Soxhlet extraction apparatus, centrifuge evaporators.

In yet a further embodiment, the membrane extract is ultra-sonicated by tip ultra-sonicator in a buffer loaded with desirable active molecules. In one embodiment, the average particle size is 0.4 pm to 1.5 pm when particle size is measured within few hours after the preparation. In yet another embodiment, the particle size is 0.8 pm to 5 pm when particle size is measured within a month after the preparation and bioxome particles are stored at 0° C. to −4° C.

In one embodiment, the bioxome particles are derived from membranes of cellular or extracellular source. In one embodiment, the bioxome particles are engineered on-demand from a pre-defined source. In one embodiment, the cell-source is autologous. The term “autologous” refers to a situation when the donor and the recipient are the same. In one embodiment, the cell-source is non-autologous. In one embodiment the donor source is mesoderm cells including, but not limited to fibroblasts, mesenchymal stem cells, pluripotent and differentiated stem cells, cells of the immune system, dendritic cells, ectoderm, keratinocytes, cells of GI and oral cavity, nasal mucosal cells, neuronal and retinal cells, endothelial cells, cardiospheres, cardiomyocytes, pericytes, and blood cells. In one embodiment, the source for the bioxome particles is stromal cells, keratinocytes, melanocytes, parenchymal cells, mesenchymal stem cells (lineage committed or uncommitted progenitor cells), liver reserve cells, neural stem cells, pancreatic stem cells, and/or embryonic stem cells, bone marrow, skin, liver tissue, pancreas, kidney tissue, neurological tissue, adrenal gland, mucosal epithelium, and smooth muscle.

In some erbodirnents, the comnposition disclosed herein is administered together with platelet-derived extracellular vesicles (EVs) or mimics of thereof.

In one embodiment, bioxomes are loaded with ribonucleases during extraction. In yet another embodiment, the loading is performed during drying, prior to extraction or post. In one embodiment, the obtained bioxome particles may undergo extrusion. In some embodiments, bioxomes are extracted by the HIP extraction system. The advantage of the HIP extraction system of the invention is that in contrast to classic chloroform-methanol lipid extraction, enables extract membrane lipids with minimal lipase activity and directly from/on chloroform-soluble components, such as plastics, cell culture sterile surface wells, including but not limited to hollow fiber, beads, nucleopore, and polycarbonate filters.

For example, HIP would permit direct extraction from polycarbonate is stable in these solvents. HIP extraction can be used for consolation of bioxomes from cells or conditioned medium in parallel with coextraction of RNA or proteins from same cell culture or tissue sample. For such process to cell layer or cell pellet or lyophilized conditioned medium or tissue extract HIP can be premixed with approx ¼-⅕ th per volume of water buffer or RNA or DNA or protein stabilizing solution (e.g. RNAsave or Trhaloze or RNAse inhibitor containing buffer). The water phase buffer or stabilizing solution extracts coprecipitated nucleic or protein extract wherein said coextracted nucleic or protein phase then may be separated for example by centrifugation or freezing gradient etc. Such RNA or/and DNA or/and protein containing phase may be further during particle formation with hydrophobic phase of bioxome particle and then used as biotherapeutics or for biomarker diagnostic or research reagent use.

In one embodiment, the process of the invention is compatible with GMP and GLP guidance. In one embodiment, according to the process of the invention, the bioxome particles are harvested from cell biomass; cellular pellet; adhesive cellular layer; medium; or a combination thereof. In one embodiment, the bioxome particles are extracted by single low-toxicity step that allow OECD approved-solvent extraction process. In one embodiment, source cells can be modified prior to the extraction by exposure to mild oxidative stress, starvation, radiation or other in vitro modification of cells in culture, in culture to express more lipophilic antioxidants. In one embodiment the lipophilic anti oxidant is rutin, squalene, tocopherol, retinol, folic acid and derivatives thereof. In one embodiment, the lipid solution component is filter-sterilized. In yet further embodiment, the lipid solution component can be stored in nitrogen or argon at a temperature of −20° C. to −80° C.

In a further embodiment, the solvent further comprises detergent surfactant. In one embodiment, the detergent is Polaxomer. In one embodiment, the process comprises lyophilizing/evaporating HIP solvent portion to form a bioxome particle-nucleic acid complex; and ultrasonicating in a hydrophilic carrier/buffer, and/or optional extrusion with desired particle size.

In the embodiments of the invention, the QC specifications for particle size characterization of bioxome particles include, without limitation, the following: particle size; penetration capacity to the target tissues/cells; sterility; non-immunogenicity and safety defined by absence of proteins and nucleic acids. Particle size distribution is measured on Malvern Nano Zetasizer and refined by Zetasizer software. The size of the bioxome particles assemblies are manipulated based on the desired application, making use of commonly available down-sizing technigues. The assemblies may be down-sized by extrusion through membranes with preselected mesh dimensions.

In the context of the invention, the QC specifications for bioxome particles lipid characterization include, without limitation, the following: bioxome particles are qualified and quantified by membrane lipid composition and characteristics, such as: (1) de/saturation index of fatty acids-FA, (2) FA chain length characteristics, (GC; HPLC analytical methods) i.e. Long chain LC-polyunstarurated FA PUFA/medium chain-MC/; (3) polarity (IZON assay); (4) lipid composition, i.e. Content percentage ad/or ratio, e.g. PL-phospholipid composition and ration PC-PE/PI-PS or ratio/percentage between various lipid groups of the Bioxome membranes, e.g. PL/NL (neutral lipid)/CL/GL/TG/FFA (HPLC; TLC; LC-MS; MALDI; column chromatography; etc.); (5) total lipid (vanillin assay, etc.); (6) optional functional lipids and lipid derivatives content, e.g. prostaglandins, prostacyclines, leukotriens, tromboxanes (HPLC; MS-MS; ELISA; RIA; etc.), or (7) metabolites such as hydroxy index- (iodine assay); and (8) ROS mediated oxidation.

In the context of the invention, the QC specifications for final composition comprising bioxome particles include, without limitation, the following: viscosity and osmolarity; pH; number of particles per batch; turbidity; stability specification parameters. Methods of particle measurements and characterization that are provided by IZON Ltd., are also applicable for QC in bioxome particles production.

In the context of the invention, the QC specifications for the bioxome production potency include, without limitation an assay for desired bioxomes activity. For example, cell culture assay to test bioxome and redoxome based products functional effect in vitro. The effect may be screened as QC potency assay by scratch assay, cytotoxicity assay, for example chemotherapeutic drug cytotoxicity assay, ROS generating or hydroxyurea aging inducing assay, inflammation IL19 or TGF beta inducing assay.

### Immune Cells

In some embodiments, the compositions disclosed herein comprise immunoglobulins, fragments thereof, antibodies, or combinations thereof, obtained from a plasma of a subject immune to a viral disease, and immune cells. In some embodiments, the compositions disclosed herein comprise plasma of a subject immune to a viral disease, and immune cells. In some embodiments, said viral disease comprise Covid-19.

A skilled artisan would appreciate that immune cells, leukocytes, or white blood cells, comprise cells of the immune system that are involved in protecting the body against both infectious disease and foreign invaders. All white blood cells are produced and derived from multipotent cells in the bone marrow known as hematopoietic stem cells.

In some embodiments, an immune cell is selected from the group comprising neutrophils, eosinophils (acidophiles), basophils, lymphocytes, and monocytes. In some embodiments, a neutrophil is selected from the group comprising segmented neutrophils and banded neutrophils.

In some embodiments, an immune cell comprises a B cell. In some embodiments, an immune cell comprises a memory B cell. In some embodiments, an immune cell comprises a regulatory B cell (Breg). In some embodiments, an immune cell comprises a T cell. In some embodiments, an immune cell comprises a Killer T cell, or cytotoxic T cell. In some embodiments, an immune cell comprises a Helper T cell. In some embodiments, an immune cell comprises a Th1 cell. In some embodiments, an immune cell comprises a Th2 cell. In some embodiments, an immune cell comprises a Regulatory T cell (Treg).

In some embodiments, an immune cell comprises a memory T cell. In some embodiments, an immune cell comprises a Natural Killer (NK) cell. In some embodiments, an immune cell comprises a monocyte. In some embodiments, an immune cell comprises a dendritic cell. In some embodiments, an immune cell comprises a macrophage. In some embodiments, an immune cell comprises a Myeloid dendritic cell (mDC). In some embodiments, an immune cell comprises a plasmacytoid dendritic cell (pDC). In some embodiments, the compositions disclosed herein comprise more than one type of immune cell.

In some embodiments, the compositions disclosed herein comprise immunoglobulins, fragments thereof, antibodies, or combinations thereof, obtained from a plasma of a subject immune to Covid-19, and NK cells. In some embodiments, disclosed herein is a composition for treating or preventing a Covid-19 in a subject comprising plasma of a subject immune to Covid-19, and NK cells.

A skilled artisan would appreciate that NK cells can be identified and isolated, for example, by cell surface markers comprising CD16 (FcTRIII), CD57, NKp46. Further, several methods are disclosed in the literature that teach how to isolate and growth NK cells. Any of these methods can be used to produce NK cells for the compositions and methods disclosed herein.

In some embodiments, immune cells comprise human immune cells. In some embodiments, immune cells are obtained from a cell line. In some embodiments, immune cells are obtained from a donor. In some embodiments, immune cells are obtained from a subject immune to a viral disease. In some embodiments, immune cells are obtained from a subject immune to Covid-19.

In some embodiments, disclosed herein is a composition for treating or preventing a viral disease, for example Covid-19 in a subject, comprising stem cells obtained from a plasma of a subject immune to said viral disease.

In some embodiments, stem cells are derived from, liver tissue, adipose tissue, bone marrow, skin, placenta, umbilical cord, Wharton's jelly or cord blood. By “umbilical cord blood” or “cord blood” is meant to refer to blood obtained from a neonate or fetus, most preferably a neonate and preferably refers to blood which is obtained from the umbilical cord or the placenta of newborns.

In some embodiments, a stem cell comprises a mesenchymal stem cell (MSC). These cells can be obtained according to any conventional method known in the art. MSC are defined by expression of certain cell surface markers including, but not limited to, CD 105, CD73 and CD90 and ability to differentiate into multiple lineages including osteoblasts, adipocytes and chondroblasts. MSC can be obtained from tissues by conventional isolation techniques such as plastic adherence, separation using monoclonal antibodies such as STRO-1 or through epithelial cells undergoing an epithelial-mesenchymal transition (EMT).

In some embodiments, adipose tissue-derived stem cells encompass undifferentiated adult stem cells isolated from adipose tissue and may also be term “adipose stem cells”, having all the same qualities and meanings. These cells can be obtained according to any conventional method known in the art.

In some embodiments, placental-derived stem cells encompass undifferentiated adult stem cells isolated from placenta and may be referred to herein as “placental stem cells”, having all the same meanings and qualities.

In some embodiments, stem cells comprise a hematopoietic stem cells (HSCs), which are the stem cells that give rise to other blood cells by haematopoiesis. In some embodiments, HSCs comprises Colony-forming unit-granulocyte-erythrocyte-monocyte-megakaryocyte (CFU-GEMM), Colony-forming unit-lymphocyte (CFU-L), Colony-forming unit-erythrocyte (CFU-E), Colony-forming unit-granulocyte-macrophage (CFU-GM), Colony-forming unit-megakaryocyte (CFU-Meg), Colony-forming unit-basophil (CFU-B), Colony-forming unit-eosinophil (CFU-Eos), or a combination thereof.

Several methods for isolating HSCs are disclosed in the literature and are thus available to a skilled artisan. Hematopoietic stem cells can be identified or isolated by the use of flow cytometry where the combination of several different cell surface markers (particularly CD34) is used to separate the rare Hematopoietic stem cells from the surrounding blood cells. Hematopoietic stem cells lack expression of mature blood cell markers and are thus, called Lin-. Lack of expression of lineage markers is used in combination with detection of several positive cell-surface markers to isolate Hematopoietic stem cells. In addition, Hematopoietic stem cells are characterised by their small size and low staining with vital dyes such as rhodamine 123 (rhodamine lo) or Hoechst 33342 (side population).

### Immunoglobulins

In some embodiments, the compositions disclosed herein further comprise immunoglobulins, fragments thereof, antibodies, or combinations thereof, obtained from a plasma of a subject immune to a viral disease. In some embodiments, the compositions disclosed herein further comprise plasma of a subject immune to a viral disease. In some embodiments, said viral disease is Covid-19, said immunoglobulins bind SARS-CoV-2, or a combination thereof.

Methods available in the art allow identifying subjects who are infected with a virus, for example CoV or SARS-CoV-2, and recover, mount, or will have mounted, an immune response to these viruses and make IgG or IgM antibodies against them. In one embodiment, these individuals are immune to the viral disease, for example Covid 2019. As a result, their plasma is used in another embodiment as a therapeutic agent to prevent said viral disease, respectively, infection in individuals who are not immune, or as treatment in those subjects who are ill with the disease. In one embodiment, the plasma of immune individuals with immunity to a viral disease is processed to manufacture an immunoglobulin preparation which is effective in preventing and/or treating said viral disease or infection, respectively.

In one embodiment, the SARS-CoV-2 immunoglobulins described herein will supply critical SARS-CoV-2 antibodies, fragments thereof or combinations thereof to subjects who are at risk for this infection. In another embodiment said anti SARS-CoV-2 antibodies, fragments thereof or combinations thereof will be administered to patients who are already ill as a result of this infection.

In one embodiment, the compositions and methods of the invention requires the collection of plasma from subjects who have been exposed to the virus, for example SARS-CoV-2, fragments thereof, its antigen(s), or combinations thereof and the use of said plasma as a therapeutic agent, or further processing of said plasma into therapeutic materials such as immunoglobulins or hyperimmune immunoglobulin preparations, in another embodiment. In one embodiment, the immunoglobulins used in the methods and compositions of the invention, are antibodies, IgG, IgM or a combination thereof.

In one embodiment, the term “antibody” includes complete antibodies (e.g., bivalent IgG, pentavalent IgM), or fragments of antibodies which contain an antigen binding site. Such fragments include in one embodiment Fab, F(ab′)2, Fv and single chain Fv (scFv) fragments. In one embodiment, such fragments may or may not include antibody constant domains. In another embodiment, Fab's lack constant domains which are required for complement fixation. ScFvs are composed of an antibody variable light chain (VL) linked to a variable heavy chain (VH) by a flexible hinge. ScFvs are able to bind antigens and can be rapidly produced in bacteria.

In some embodiments, the current disclosure further includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (VH) and light chain variable domains (VL) which together comprise Fv or scFv, with the addition, in another embodiment, of a heavy chain constant domain (CH1) and a light chain constant domain (CL). The four domains (i.e., VH-CH1 and VL-CL) comprise a Fab. Complete antibodies are obtained in one embodiment, from such a library by replacing missing constant domains once a desired VH-VL combination has been identified.

In one embodiment, the antibody, a fragment thereof, or combinations thereof have sufficiently high affinity and avidity to their target, which may be a viral protein, a peptide, a nucleic acid, a sugar or a combination thereof. In one embodiment the target may be CoV or SARS-CoV-2, or fragments of CoV or SARS-CoV-2, or a combination thereof.

In another embodiment, fractionating the plasma sample, the sample with the immunoglobulins fragments thereof, anti-virus antibodies, or combinations thereof, comprises amplifying the target gene encoding for immunoglobulins fragments thereof, anti-virus antibodies, or combinations thereof. In one embodiment, the terms “amplification” or “to amplify” refer to one or more methods known in the art for copying a target nucleic acid, thereby increasing the number of copies of a selected nucleic acid sequence. Amplification may be exponential in one embodiment, or linear in another. In one embodiment, a target nucleic acid may be either DNA or RNA. The sequences amplified in this manner form an “amplicon.” While the exemplary embodiments described herein relate to amplification using the polymerase chain reaction (“PCR”), numerous other methods are known in the art for amplification of nucleic acids (e.g., isothermal methods, rolling circle methods, etc.) and are considered within the scope of the present invention. The skilled artisan will understand that these other methods may be used either in place of, or together with, PCR methods.

In another embodiment, real time PCR is used in the methods of the invention. The term “real time PCR” refers in one embodiment to the process where a signal emitted from the PCR assay is monitored during the reaction as an indicator of amplicon production during each PCR amplification cycle (i.e., in “real time”), as opposed to conventional PCR methods, in which an assay signal is detected at the endpoint of the PCR reaction. Real time PCR is based in one embodiment on the detection and quantitation of a virus reporter, for example a SARS-CoV-2 reporter. The signal increases in direct proportion to the amount of PCR product in a reaction. By recording the amount of the virus reporter at each cycle, it is possible to monitor the PCR reaction during exponential phase where the first significant increase in the amount of PCR product correlates to the initial amount of target template.

The prevalence of antibodies to a virus, for example SARS-CoV-2, varies considerably among different populations. Plasma will be collected in one embodiment from healthy subjects who have been previously exposed to the virus, for example SARS-CoV-2, either naturally in one embodiment, or by deliberate vaccination (immunization) in another embodiment, and who have antibodies to the virus in their plasma. These subjects are ascertained in one embodiment from populations where viral infection is high, who have a history of viral infections in the past, who are found to have antibodies to the virus thorough an antibody screening program, who have antibodies as the result of deliberate immunization with the virus or with antigens associated with the virus, or a combination thereof.

The processing of subjects (“donors”) shall conform to the regulatory requirements that are applicable in the jurisdiction(s) in which the collections take place. This includes soliciting a medical history and measuring pre-donation parameters (such as blood pressure, temperature, hemoglobin, etc.). In another embodiment, after each donation the collected plasma is screened for markers for transmissible disease (e.g. anti-HIV, anti-HCV, HBsAg, Syphilis, etc.) that are applicable in the jurisdiction(s) in which the collections take place, to minimize the hazard of disease transmission. In one embodiment, all donors are screened for the presence of antibodies to the virus, for example SARS-CoV-2, and in another embodiment, the quantity of antibodies is ascertained.

In one embodiment, the plasma used in the methods and compositions of the invention will be collected from a subject by either plasmapheresis (as source plasma) or after separation from whole blood donations (as recovered plasma). In one embodiment, “plasmapheresis” refers to a process in which the part of the blood, is removed from blood cells by a cell separator. The separator works by either spinning the blood at high speed to separate the cells from the blood, or by passing the blood through a membrane with a cellular sieve, so that only the plasma can pass through. The cells are returned in one embodiment to the person undergoing treatment, while the plasma, which contains the antibodies, is collected.

In one embodiment, the term “recovered plasma” refers to the plasma that is, or has been, separated from whole blood donations. In another embodiment, “recovered plasma” refers to the process whereby heparinized blood is passed through the first filter of a cascade consisting of several filters into a stream containing the corpuscular components and a plasma stream, subjecting the plasma stream to a purification process, recombining the purified plasma and the stream containing the corpuscular particles and reinfusing the recombined blood into the subject. In one embodiment, the purified plasma is recovered, and IgG, IgM, antibodies, their fragments or antigens are removed prior to the recombination of the plasma and the stream containing the corpuscular particles.

After collection, the plasma is frozen in one embodiment, or stored in the liquid state for an appropriate period of time in another embodiment. Conditions of storage will be determined on the basis of optimal preservation of the anti-viral antibodies as well as preventing contamination of the plasma. In one embodiment, usual (frozen) storage and shipping conditions that are applicable to other plasma products are employed for the antibody plasma preparation.

In one embodiment, a concentrated hyperimmune globulin appropriate for use in the treatment or prevention of a viral disease will be prepared from the collected plasma. In another embodiment, the plasma will be pooled in appropriately-sized batches and subjected to a plasma fractionation procedure which will isolate in one embodiment, and/or purify the immunoglobulin fraction and/or anti-viral antibodies from the plasma in other embodiments. This is done in one embodiment by the classical Cohn alcohol precipitation method, or a variant thereof, an ion exchange chromatographic method, an affinity chromatographic method, or any other suitable method such as MS-MS (tandem mass spectrometry), LC-MS (preparatory liquid chromatography and mass spectrometry), crystallization or immunopercipitation methods etc. in other embodiments. The final material will be concentrated and the titer or quantity of anti-viral antibody adjusted as appropriate. The final material will be sterile and will meet regulatory requirements as applicable in the jurisdiction of manufacture and/or use.

According to this aspect of the invention and in one embodiment, provided is a method of producing a pharmaceutical preparation for the prevention or treatment of a viral disease, for example Covid-2019, comprising: obtaining plasma from a subject immune to the viral disease; pooling said plasma; fractionating said plasma wherein said fractionation isolates or purifies an immunoglobulin, a fragments thereof, an anti-viral antibody, or a combination thereof from the plasma; and concentrating said immunoglobulin, fragments thereof, antibody, or combinations thereof.

In one embodiment, the final material may have a protein concentration of 0.5%-15%. In one embodiment, the protein concentration is between 0.1 and about 1% (w/w) or between about 1 and about 5% (w/w) in another embodiment, or between about 5 and about 10% (w/w) in another embodiment, or between about 10 and about 15% (w/w) in another embodiment. The final formulation may be appropriate for either intravenous, intrapulmonary, intracavitary or intramuscular administration, or both. Shelf life of the materials is ascertained in one embodiment, through appropriate stability studies.

The efficacy of all immunization programs for the prevention and treatment of bacterial or viral infections is based in one embodiment, on the magnitude of circulating antibody levels. Dosing schedules and product specifications are constructed in certain embodiments around the level of antibodies that is generated (in the case of active immunization in one embodiment) or administered (in the case of passive immunization in other embodiments). In one embodiment, Intravenous Immune Globulins (IVIG) are used in patients with primary immune deficiency. These patients are born with hypo- or agammaglobulinemia and are at great risk for life-threatening infection. The life-long monthly administration of IVIG, however, affords these patients a high level of protection against bacterial and viral infections and permits them to live a normal life by providing them, passively, with a broad array of antibody specificities present in a large number of plasmapheresis donors from which the IVIG was manufactured.

### Viral Diseases

In some embodiments, a viral disease is caused by SARS-CoV-2. In some embodiments, a viral disease is caused by an adenovirus. In some embodiments, a viral disease is caused by a herpesvirus. In some embodiments, a viral disease is caused by a papillomavirus. In some embodiments, a viral disease is caused by a polyomavirus. In some embodiments, a viral disease is caused by a poxvirus. In some embodiments, a viral disease is caused by an hepadnavirus. In some embodiments, a viral disease is caused by a parvovirus. In some embodiments, a viral disease is caused by an astrovirus.

In some embodiments, a viral disease is caused by a calicivirus. In some embodiments, a viral disease is caused by a picornavirus. In some embodiments, a viral disease is caused by a coronavirus. In some embodiments, a viral disease is caused by a flavivirus. In some embodiments, a viral disease is caused by a togavirus. In some embodiments, a viral disease is caused by a hepevirus. In some embodiments, a viral disease is caused by a retrovirus. In some embodiments, a viral disease is caused by an orthomyxovirus. In some embodiments, a viral disease is caused by an arenavirus. In some embodiments, a viral disease is caused by a bunyavirus.

In some embodiments, a viral disease is caused by a filovirus. In some embodiments, a viral disease is caused by a paramyxovirus. In some embodiments, a viral disease is caused by a rhabdovirus. In some embodiments, a viral disease is caused by a reovirus. In some embodiments, a viral disease is caused by Herpes simplex type 1. In some embodiments, a viral disease is caused by Herpes simplex type 2. In some embodiments, a viral disease is caused by Varicella-zoster virus.

In some embodiments, a viral disease is caused by Epstein-Barr virus. In some embodiments, a viral disease is caused by Human cytomegalovirus. In some embodiments, a viral disease is caused by human herpesvirus type 8. In some embodiments, a viral disease is caused by human papillomavirus. In some embodiments, a viral disease is caused by BK virus. In some embodiments, a viral disease is caused by JC virus. In some embodiments, a viral disease is caused by smallpox. In some embodiments, a viral disease is caused by Hepatitis B virus.

In some embodiments, a viral disease is caused by parvovirus B19. In some embodiments, a viral disease is caused by human astrovirus. In some embodiments, a viral disease is caused by Norwalk virus. In some embodiments, a viral disease is caused by coxsackievirus. In some embodiments, a viral disease is caused by hepatitis A virus. In some embodiments, a viral disease is caused by poliovirus. In some embodiments, a viral disease is caused by rhinovirus. In some embodiments, a viral disease is caused by severe acute respiratory syndrome virus. In some embodiments, a viral disease is caused by hepatitis C virus. In some embodiments, a viral disease is caused by yellow fever virus.

In some embodiments, a viral disease is caused by dengue virus. In some embodiments, a viral disease is caused by West Nile virus. In some embodiments, a viral disease is caused by TBE virus. In some embodiments, a viral disease is caused by Rubella virus. In some embodiments, a viral disease is caused by Hepatitis E virus. In some embodiments, a viral disease is caused by Human immunodeficiency virus (HIV). In some embodiments, a viral disease is caused by Influenza virus. In some embodiments, a viral disease is caused by Lassa virus. In some embodiments, a viral disease is caused by Crimean-Congo hemorrhagic fever virus.

In some embodiments, a viral disease is caused by Hantaan virus. In some embodiments, a viral disease is caused by Ebola virus. In some embodiments, a viral disease is caused by Marburg virus. In some embodiments, a viral disease is caused by Measles virus. In some embodiments, a viral disease is caused by Mumps virus. In some embodiments, a viral disease is caused by Parainfluenza virus. In some embodiments, a viral disease is caused by Respiratory syncytial virus. In some embodiments, a viral disease is caused by Rabies virus.

In some embodiments, a viral disease is caused by Hepatitis D. In some embodiments, a viral disease is caused by Rotavirus. In some embodiments, a viral disease is caused by Orbivirus. In some embodiments, a viral disease is caused by Coltivirus. In some embodiments, a viral disease is caused by Banna virus. In some embodiments, a viral disease is caused by more than one virus.

In some embodiments, said viral disease comprises Covid-19. In some embodiments, said viral disease comprises acute hepatitis. In some embodiments, said viral disease comprises AIDS. In some embodiments, said viral disease comprises aseptic meningitis. In some embodiments, said viral disease comprises bronchiolitis. In some embodiments, said viral disease comprises Burkitt's lymphoma. In some embodiments, said viral disease comprises chickenpox. In some embodiments, said viral disease comprises chronic hepatitis.

In some embodiments, said viral disease comprises common cold. In some embodiments, said viral disease comprises congenital rubella. In some embodiments, said viral disease comprises congenital varicella syndrome. In some embodiments, said viral disease comprises congenital seizures in the newborn. In some embodiments, said viral disease comprises croup. In some embodiments, said viral disease comprises cystitis. In some embodiments, said viral disease comprises cytomegalic inclusion disease. In some embodiments, said viral disease comprises fatal encephalitis. In some embodiments, said viral disease comprises gastroenteritis.

In some embodiments, said viral disease comprises German measles. In some embodiments, said viral disease comprises gingivostomatitis. In some embodiments, said viral disease comprises hepatic cirrhosis. In some embodiments, said viral disease comprises hepatocellular carcinoma. In some embodiments, said viral disease comprises herpes labialis. In some embodiments, said viral disease comprises cold sores.

In some embodiments, said viral disease comprises herpes zoster. In some embodiments, said viral disease comprises Hodgkin's lymphoma. In some embodiments, said viral disease comprises hyperplastic epithelial lesions. In some embodiments, said viral disease comprises warts. In some embodiments, said viral disease comprises laryngeal papillomas.

In some embodiments, said viral disease comprises epidermodysplasia verruciformis. In some embodiments, said viral disease comprises infectious mononucleosis. In some embodiments, said viral disease comprises influenza. In some embodiments, said viral disease comprises influenza-like syndrome. In some embodiments, said viral disease comprises Kaposi sarcoma. In some embodiments, said viral disease comprises keratoconjunctivitis.

In some embodiments, said viral disease comprises liver. In some embodiments, said viral disease comprises lung and spleen diseases in the newborn. In some embodiments, said viral disease comprises malignancies. In some embodiments, said viral disease comprises cervical carcinoma. In some embodiments, said viral disease comprises squamous cell carcinomas. In some embodiments, said viral disease comprises measles. In some embodiments, said viral disease comprises multicentric Castleman disease.

In some embodiments, said viral disease comprises mumps. In some embodiments, said viral disease comprises myocarditis. In some embodiments, said viral disease comprises nasopharyngeal carcinoma. In some embodiments, said viral disease comprises pericarditis. In some embodiments, said viral disease comprises pharyngitis. In some embodiments, said viral disease comprises pharyngoconjunctival fever. In some embodiments, said viral disease comprises pleurodynia.

In some embodiments, said viral disease comprises pneumonia. In some embodiments, said viral disease comprises poliomyelitis. In some embodiments, said viral disease comprises postinfectious encephalomyelitis. In some embodiments, said viral disease comprises premature delivery. In some embodiments, said viral disease comprises primary effusion lymphoma. In some embodiments, said viral disease comprises rabies. In some embodiments, said viral disease comprises Reye syndrome.

In some embodiments, said viral disease comprises severe bronchiolitis with pneumonia. In some embodiments, said viral disease comprises skin vesicles. In some embodiments, said viral disease comprises mucosal ulcers. In some embodiments, said viral disease comprises tonsillitis. In some embodiments, said viral disease comprises pharyngitis.

A skilled artisan will recognize that Covid-19, also termed “novel coronavirus pneumonia”, “NCP”, “SARS-CoV-2 acute respiratory disease”, and “COVID-19” comprises an infectious respiratory disease caused by the 2019 novel coronavirus (SARS-CoV-2), which was first detected during the 2019-20 Wuhan coronavirus outbreak. In some embodiments, SARS-CoV-2 is transmitted through human-to-human transmission, generally via respiratory droplets as sneeze, cough or exhalation. In some embodiments, NCP symptoms appear after an incubation period of between 2 to 14 days. In some embodiments, coronavirus primarily affects the lower respiratory tract. In some embodiments, coronavirus primarily affects the upper respiratory tract. In some embodiments, NCP symptoms comprise fever, coughing, shortness of breath, pain in the muscles, tiredness, pneumonia, acute respiratory distress syndrome, sepsis, septic shock, death, or any combination thereof.

A skilled artisan will recognize that SARS-CoV-2 belongs to the broad family of viruses known as coronaviruses. SARS-CoV-2 is a positive-sense single-stranded RNA (+ssRNA) virus. SARS-CoV-2 is a member of the subgenus Sarbecovirus (Beta-CoV lineage B), having an RNA sequence of approximately 30,000 bases in length.

Eighty-one genomes of SARS-CoV-2 had been isolated and reported. The present disclosure comprises compositions and methods for treating these SARS-CoV-2 variants, or any further one.

A skilled artisan will recognize that seven coronavirus types are known to affect humans. The compositions and methods disclosed herein are useful for treating any of them. In some embodiments, coronavirus comprises Human coronavirus 229E (HCoV-229E). In some embodiments, coronavirus comprises Human coronavirus OC43 (HCoV-OC43). In some embodiments, coronavirus comprises Severe acute respiratory syndrome-related coronavirus (SARS-CoV). In some embodiments, coronavirus comprises Human coronavirus NL63 (HCoV-NL63, New Haven coronavirus). In some embodiments, coronavirus comprises Human coronavirus HKU1. In some embodiments, coronavirus comprises Middle East respiratory syndrome-related coronavirus (MERS-CoV), previously known as novel coronavirus 2012 and HCoV-EMC. In some embodiments, coronavirus comprises Novel coronavirus (SARS-CoV-2), also known as Wuhan coronavirus.

In some embodiments, diseases related to CoV comprise common cold, pneumonia, viral pneumonia or a secondary bacterial pneumonia, bronchitis, direct viral bronchitis or a secondary bacterial bronchitis, severe acute respiratory syndrome (SARS), and Middle East respiratory syndrome (MERS).

In some embodiments, the compositions disclosed herein are used to treat or prevent SARS. In some embodiments, the compositions disclosed herein are used to treat or prevent MERS. In some embodiments, the compositions disclosed herein are used to treat or prevent HPV. In some embodiments, the compositions disclosed herein are used to treat or prevent HIV. In some embodiments, the compositions disclosed herein are used to treat or prevent Ebola.

### Methods of Treatment

In some embodiments, the invention provides a method of treating a viral disease in a subject in need thereof, the method comprising administering a composition comprising a ribonuclease and a bioxome, an exosome, or a combination thereof. In one embodiment, the method comprises administering a composition comprising ranpirnase and a bioxome, an exosome, or a combination thereof. In another embodiment, the method comprises administering a composition comprising ranpirnase and a bioxome. In another embodiment, the method comprises administering a composition comprising ranpirnase and an exosome.

In some embodiments, the invention provides a method of treating a viral disease in a subject in need thereof, the method comprising administering any of the compositions as describes herein. In some embodiments, the invention provides a method of treating any viral disease in a subject in need thereof, as described herein. In some embodiments, the invention provides a method of treating a viral disease in a subject in need thereof, wherein the viral disease is caused by any virus as disclosed herein.

In some embodiments, disclosed herein is a composition for treating or preventing a viral disease in a subject, said composition comprising ribonuclease. In some embodiments, disclosed herein is a composition for treating or preventing a Covid-19 in a subject, said composition comprising ribonuclease.

In some embodiments, disclosed herein is a composition for treating or preventing a viral disease in a subject, said composition comprising immunoglobulins, fragments thereof, antibodies, or combinations thereof, obtained from a plasma of a subject immune to said viral disease. In some embodiments, disclosed herein is a composition for preventing Covid-19 in a subject, said composition comprising immunoglobulins, fragments thereof, antibodies, or combinations thereof, obtained from a plasma of a subject immune to Covid-19.

In some embodiments, disclosed herein is a composition for treating or preventing a viral disease in a subject, said composition comprising immune cells. In some embodiments, disclosed herein is a composition for treating or preventing a Covid-19 in a subject, said composition comprising immune cells.

In some embodiments, disclosed herein is a composition for treating or preventing a viral disease in a subject, said composition comprising a ribonuclease and immunoglobulins, fragments thereof, antibodies, or combinations thereof, obtained from a plasma of a subject immune to said viral disease. In some embodiments, disclosed herein is a composition for treating or preventing a Covid-19 in a subject, said composition comprising a ribonuclease and immunoglobulins, fragments thereof, antibodies, or combinations thereof, obtained from a plasma of a subject immune to Covid-19.

In some embodiments, disclosed herein is a composition for treating or preventing a viral disease in a subject, said composition comprising a ribonuclease and immune cells. In some embodiments, disclosed herein is a composition for treating or preventing a Covid-19 in a subject, said composition comprising a ribonuclease and immune cells.

In some embodiments, disclosed herein is a composition for treating or preventing a viral disease in a subject, said composition comprising a ribonuclease, immunoglobulins, fragments thereof, antibodies, or combinations thereof, obtained from a plasma of a subject immune to said viral disease, and immune cells. In some embodiments, disclosed herein is a composition for treating or preventing a Covid-19 in a subject, said composition comprising a ribonuclease, immunoglobulins, fragments thereof, antibodies, or combinations thereof, obtained from a plasma of a subject immune to Covid-19, and immune cells.

In some embodiments, disclosed herein is a composition for treating or preventing a viral disease in a subject, said composition comprising a immunoglobulins, fragments thereof, antibodies, or combinations thereof, obtained from a plasma of a subject immune to said viral disease, and immune cells. In some embodiments, disclosed herein is a composition for treating or preventing a Covid-19 in a subject, said composition comprising immunoglobulins, fragments thereof, antibodies, or combinations thereof, obtained from a plasma of a subject immune to Covid-19, and immune cells.

In some embodiments, disclosed herein is a composition for treating or preventing a viral disease in a subject, said composition comprising immunoglobulins, fragments thereof, antibodies, or combinations thereof, obtained from a plasma of a subject immune to a viral disease, ribonuclease-loaded bioxomes, and immune cells. In some embodiments, disclosed herein is a composition for treating or preventing a Covid-19 in a subject, said composition comprising immunoglobulins, fragments thereof, antibodies, or combinations thereof, obtained from a plasma of a subject immune to a viral disease, ribonuclease-loaded bioxomes, and immune cells.

In one embodiment, disclosed herein is a composition for treating or preventing a viral disease in a subject, said composition comprising a ribonuclease. In another embodiment, the composition is loaded into a bioxome, an exosome, or a combination thereof. In another embodiment, the composition is administered with a bioxome, an exosome, or a combination thereof.

Two forms of immunization have been utilized with great success for more than 50 years both for the treatment and prevention of bacterial and viral infections. These are termed active and passive immunization. In some embodiments, the compositions disclosed herein can be used for passive and active immunization.

In one embodiment, active immunization (also called vaccination) involves the administration of either a live, attenuated or killed microorganism, or a portion of said microorganism in order “prime” the cellular immune system and to elicit an antibody response in the subject. Microoganisms may be a baterium, a virus, a virus-like particle or a combination thereof. The antibody response-which results in certain embodiments, is the ability of the subject's immune system to select, synthesize and secrete antibodies that will kill the specific invading microorganism-takes some weeks or months to occur, during which time the subject remains vulnerable to the microorganism. However, once vaccinated, the subject retains the ability to defend himself against that microorganism for part or the rest of his or her life, at least in part by raising specific antibodies against the microorganism when exposed. (although booster immunizations may be required periodically). Active immunization has been shown to be highly effective in conferring long-term protection against certain conditions and is generally administered when the subject is well and has not been recently exposed to the innoculum. Examples of active viral vaccines include smallpox, polio, and hepatitis B.

Passive immunization involves in another embodiment, the administration to the subject of a purified immunoglobulin preparation which contains relatively high quantities of one or more antibodies specific to the target microorganism. In one embodiment, passive administration of such antibodies confers immediate but temporary immunity against a specific microorganism, usually for the time that the antibodies are present in the body (perhaps a month or two). As a result, passive immunization is used when the subject has been recently exposed to a specific microorganism or is at high risk of being exposed to a microorganism in an attempt to prevent, or modify the severity of, disease caused by the microorganism in question. Examples of viral passive antibodies given prophylactically include Rabies immunoglobulin and Varicella-Zoster immunoglobulin. In some cases, passive immunization is given when the subject is already ill, as a therapeutic agent. Examples of passive immunization include but are not limited to viral antibodies given therapeutically, include Hepatitis B immunoglobulin [in liver transplants for Hepatitis B liver failure and Cytomegalovirus immunoglobulin. These therapies have proven to be highly effective as well.

In one embodiment, the compositions of the invention are used in the methods of the invention described herein. In one embodiment, the invention provides a method of preventing or treating a viral disease, for example Covid-2019 in a subject, comprising any of the compositions disclosed herein. In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combinations thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

As used herein, “subject” refers in one embodiment, to a human or any other animal which has been exposed to and is now immune to CoV related disease or Covid-2019. A subject refers to a human presenting to a medical provider for diagnosis or treatment of a disease, such as a CoV related disease or Covid-2019 in another embodiment. A human includes pre- and postnatal forms. In one embodiment, subjects are humans being treated for symptoms associated with a CoV related disease or Covid-2019, or a volunteer for hyperimmune antibody production following the volunteer's exposure to an attenuated virus or the like.

In some embodiments, an extracorporeal device is used to deliver the pharmaceutical composition. In some embodiments, the patient blood is previously cleaned by antiviral phototherapy. In some embodiments, phototherapy comprises antiviral agents such as methylene blue, rose Bengal, carbon dot, quantum dot, activated photosensors or a combination thereof. In some embodiments, plasmapheresis comprises cutting off viral particles with TTF ultrafiltration or by hollow fiber filtration exposed to carbon dot and/or other phototherapy synergistic enhancers.

The term “therapeutically effective amount” or “effective amount” refers in one embodiment, to an amount of a monovalent or combination vaccine sufficient to elicit a protective immune response in the subject to which it is administered. The immune response may comprise, without limitation, induction of cellular and/or humoral immunity.

The amount of a vaccine that is therapeutically effective may vary depending on the particular antibody used in the vaccine, the age and condition of the subject, and/or the degree of infection, and can be determined by an attending physician.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used. In another embodiment, polymeric materials can be used. In another embodiment, a controlled release system can be placed in proximity to the therapeutic target, i.e., the brain, thus requiring only a fraction of the systemic dose.

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexion with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, virosomes, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral, as well as self-administration devices.

The dosage regimen for treating a condition with the compositions of this invention is selected in one embodiment, in accordance with a variety of factors, such as the type, age, weight, ethnicity, sex and medical condition of the subject, the severity of the condition treated, the route of administration, and the particular compound employed, and thus may vary widely while still be in the scope of the invention.

### Pharmaceutical Compositions

In one embodiment, the pharmaceutical preparation of the invention, used in the methods of the invention comprise a carrier, excipient, flow agent, processing aid, a diluent, or a combination thereof. In another embodiment, the pharmaceutical composition comprises the composition as described above and an excipient.

In one embodiment, the compositions used in the invention further comprise a carrier, or excipient, lubricant, flow aid, processing aid or diluent in other embodiments, wherein the carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetener, a film forming agent, or any combination thereof.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal opthalmic and oral. In one embodiment the pharmaceutical composition is administered parenterally, transmucosally, transdermally, intramuscularly, intravenously, intradermally, subcutaneously, intraperitonealy, intraventricularly, or intracranially.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intratracheal, intranasal, pulmonary, intramuscular, subcutaneous, parenteral, intraperitoneal, intracranial, transmucosal, transdermal, subcutaneous, topical administration or any combination thereof. In one embodiment the composition is a controlled release composition. In another embodiment, the composition is an immediate release composition. In one embodiment, the composition is a liquid dosage form. In another embodiment, the composition is a solid dosage form.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations of the invention can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be affected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In some embodiments, the compositions disclosed herein are encapsulated within extracellular vesicles (Evs), or mimics thereof, or submicron particles, according to prior art known technologies. In some embodiments, the pharmaceutical composition is formulated as a sterile lyophilizate. In some embodiments, the lyophilizate of immunoglobulins is preferably encapsulated within extracellular vesicles—Evs, or mimics thereof, or submicron particles.

In some embodiments, prior to administration, the lyophilizate is resuspended aseptically in a sterile buffer. In some embodiments, a physiologically tolerated buffer is added to facilitate pH control. In some embodiments, the formulations of the present invention have pH between about 6.8 and about 7.8. In some embodiments, buffers include phosphate buffers, sodium phosphate, or phosphate buffered saline (PBS). In some embodiments, the final product lyophilizate may include preformulated isotonicity agents as glycerin, stabilizers excipients, such as carbohydrates (trehaloze, sucrose), an antioxidant, a chelating agent, such as EDTA and EGTA, human serum albumin, or a combination thereof, which can optionally be added to the formulations or compositions to reduce aggregation. Surfactants additives are particularly useful if a pump or plastic container is used to administer the formulation. An optional carrier additive is human serum albumin, or an enhancer surfactant as described below. The presence of pharmaceutically acceptable surfactant mitigates the propensity of proteins to aggregate. Such acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), block co-polymers known in the state of the art. Further, glycerol monolaurate—GML, an approved pharmaceutical excipient surfactant, can be used at concentrations up to 3 mg/ml (which is similar to the amount of GML in human milk) to prepare enhanced delivery system to penetrate cell barriers if administered orally or intramucosally.

In some embodiments, the composition disclosed herein is formulated for intravenous, oral, intranasal, pulmonary, transdermal, parenteral, intraperitoneal, intracranial, intramuscular, subcutaneous, intratracheal, or transmucosal delivery, or any combination thereof.

In some embodiments, the composition disclosed herein is administered in combination with plasrnapheresis collected from a healthy donor. In some embodiments, the composition disclosed herein is delivered by a special extracorporeal device. In some embodiments, the composition disclosed herein is administered together with platelet-derived extracellular vesicles (EVs) or mimics of thereof. In another embodiment, the composition disclosed herein is synergistically combined with cellular and acellular components obtained from placental tissue and/or placental perfusate. In some embodiments, placental cells combined with the pharmaceutical composition comprise hematopoietic (CD34.sup.+) cells, nucleated cells such as granulocytes, monocytes and macrophages, a small percentage (less than 1%) of substrate-adherent placental stem cells, and natural killer cells.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual subject, the site of delivery, the method of administration and other factors known to practitioners. Examples of techniques and protocols can be found in Remington's Pharmaceutical Sciences.

In some embodiments, a synergistic effect is attained by delivering the pharmaceutical composition by various routes of administration, such as intravenous, intravesicular, oral, nasal, intraperitoneal, intrapulmonary (inhalation), intramuscular, subcutaneous, intra-tracheal, transmucosal, or transdermal route. In some embodiments, osmotic or micro pumps are used to deliver the pharmaceutical composition by any of these routes. In some embodiments, the compositions are formulated as a gel or spray.

A skilled artisan will appreciate that in some embodiments, an optimal dose for a route of administration is determined by a biosensing complex comprising a mixture of polydopamine (PDA) and protein G. PDA is a representative mussel-inspired polymer, and protein G is an immunoglobulin-binding protein that enables an antibody to have an optimal orientation.

In one embodiment, the pharmaceutical composition disclosed herein further comprises an additional therapeutic agent, a vaccine, an adjuvant or a combination thereof.

Adjuvants suitable for use in the compositions and methods described herein include, but are not limited to several adjuvant classes such as; mineral salts, e.g., Alum, aluminum hydroxide, aluminum phosphate and calcium phosphate; surface-active agents and microparticles, e.g., nonionic block polymer surfactants (e.g., cholesterol), virosomes, saponins (e.g., Quil A, QS-21, Alum and GPI-0100), proteosomes, immune stimulating complexes, cochleates, quarterinary amines (dimethyl diocatadecyl ammonium bromide (DDA)), pyridine, vitamin A, vitamin E; bacterial products such as the RIBI adjuvant system (Ribi Inc.), cell wall skeleton of Mycobacterum phlei (Detox.®.), muramyl dipeptides (MDP) and tripeptides (MTP), monophosphoryl lipid A, Bacillus Calmete-Guerin (BCG), heat labile E. coli enterotoxins, cholera toxin, trehalose dimycolate, CpG oligodeoxnucleotides; cytokines and hormones, e.g., interleukins (IL-1, IL-2, IL-6, IL-12, IL-15, IL-18), granulocyte-macrophage colony stimulating factor, dehydroepiandrosterone, 1,25-dihydroxy vitamin D3; polyanions, e.g., dextran; polyacrylics (e.g., polymethylmethacrylate, Carbopol 934P); carriers e.g., tetanus toxid, diptheria toxoid, cholera toxin B subnuit, mutant heat labile enterotoxin of enterotoxigenic E. coli (rmLT), heat shock proteins; oil-in-water emulsions e.g., AMPHIGEN® (Hydronics, USA); and water-in-oil emulsions such as, e.g., Freund's complete and incomplete adjuvants.

In some embodiments, the pharmaceutical compositions disclosed herein can be administered with a further antiviral compound. In some embodiments, said further antiviral compounds enhance the effect of the pharmaceutical compositions synergistically.

In some embodiments, said further antiviral agents are selected from a group comprising an antiviral enzyme or an approved antiviral drug, acyclovir, valaciclovir, famciclovir, sofosbuvir, ribavirin, pegylated interferon-.alpha.-2a, pegylated interferon-.alpha., penciclovir.-2b, boceprevir, telaprevir, ledipasvir, and simiprevir, nucleoside reverse transcriptase inhibitors, non-nucleoside reverse transcriptase inhibitors, protease inhibitors, and fusion inhibitors, valomaciclovir stearate, octadecyloxyethyl-cidofovir, hexadecyloxypropyl-cidofovir, adefovir, amantadine, arbidol, brivudine, darunavir, docosanol, edoxudine, entecavir, fomivirsen, fosfonet, ibacitabine, immunovir, idoxuridine, imiquimod, inosine, loviride, raltegravir, maraviroc, moroxydine, nelfinavir, nexavir, oseltamivir, peramivir, pleconaril, podophyllotoxin, rimantidine, tenofovir, tipranavir, trifluridine, tromantidine, vicriviroc, vidarabine, viramidine, zanamivir, (2-amino-7-[(1,3-dihydroxy-2-propoxy)methyl]purine], (1′S,2′R)-9-[[1′,2′-bis(hydroxymethyl)cycloprop-1′-yl]methyl]guanine (A-5021), cyclopropavir, 2,4-diamino-6-R-[3-hydroxy-2(phosphonomethoxy)propoxy]-pyrimidine, (S)-9-(3-hydroxy-2-phosphonylmethoxypropyl)adenine (S-HPMPA), 3-deaza-9-(3-hydroxy-2-phosphonylmethoxypropyl)adenine (3-deaza-HPMPA), N-(4-chlorobenzyl)-1-methyl-6-(4-morpholinylmethyl)-4-oxo-1,4-dihydro-3-q-uinolinecarboxamine (PNU-183792), 2-bromo-5,6-dichloro-1-(.beta.-D-ribofuranosyl)benzimidazole (BDCRB), maribavir, 3-hydroxy-2,2-dimethyl-N-[4-{[(5-dimethylamino)-1-naphthyl]-sulfonyl]-ami-no)phenyl}propamide (BAY 38-4766), N—[N-[4-(2-aminothiazol-4-yl)phenyl]carbamoylmethyl]-N-[1(S)-phenylethyl-]pyridine-4-carboxamide (BILS179BS), N-[5-(aminosulfonyl)-4-methyl-1,3-thiazol-2-yl]-N-methyl-2-{4-(2-pyridiny-1)phenyl}acetamide (BAY 57-1293), 2H-3-(4-chlorophenyl)-3,4-dihydro-1,4-benzo-thiazine-2-carbonitrile 1,1-dioxide, 2-chloro-3-pyridin-3-yl-5,6,7,8-tetrahydronindolizine-1-carboxamide (CMV423), or any combination thereof.

In some embodiments, a synergistic enhancer is selected from a group comprising hydroxyurea, leflunomide, EGCG, CBD, squalamine or aminosterol, mycophenolic acid, resveratrol, or a combination thereof. In some embodiments, these synergistic enhancers enhance the effects of the pharmaceutical composition in a synergistic manner.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more. rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, in addition to the immunoglobulins fragments thereof, anti-viral antibodies, or combinations thereof, used in the pharmaceutical preparations of the invention, which in another embodiment are used in the methods of the invention, the pharmaceutical preparations comprise a vaccine comprising nucleic acids encoding polypeptides of the respective virus.

### Delivery by Aerosol Spray

In some embodiments, the compositions disclosed herein are delivered by aerosol. In some embodiments, the compositions are formulated considering the particle size distribution of aerosol used to deliver it. A skilled artisan will appreciate that the aerodynamic particle size distribution is influenced by the characteristics of the spray of the drug product and engineering parameters of the delivery device. Further, particles formulation should be designed to avoid aggregation, improve aerodynamic flowability and reach high uniformity of particle size.

Particle size below 5 microns can reach bronchi and lungs, while the particles between 5-10 microns are suitable for nasal delivery. A skilled artisan will appreciate that particles above 10 microns are usually not suitable for inhalation, as they are swallowed and go directly into gastrointestinal tract. In some embodiments, particle size is in a range of about 0.1 micron and 5 micron. In some embodiments particle size is in a range of about 0.2 micron and 1 micron. In some embodiments, a range of about 0.2 micron and 1 micron allows the particles to reach the lungs. In some embodiments, particle size is in a range of about 0.5 micron and 1.5 micron. In some embodiments, a range of about 0.5 micron and 1.5 micron allows the particles to be absorbed by transmucosal delivery.

Glycerol monolaurate (GML) is a natural surfactant permeability enhancer that can 5 be used for transmucosal delivery of ribonucleases to enhance its anti-viral bioactivity. In one embodiment, GML is used to improve encapsulation of hydrophilic ranpirnase. In some embodiment, GML is used to improve efficacy of ranpirnase. In some embodiments, GML is used at its critical micelles concentrations to prepare the composition in the presence of carbohydrate/HSA stabilizers an aqueous base. In some embodiments, the size of these nanospheres for transmucosal delivery is smaller than 100 nm.

In some embodiments, a protein compatible solvent comprising an optional gamma irradiation dose (5-20 kGy at the rate of more than 1 kGy per hour) is used for the preparation of the compositions disclosed herein. In some embodiments, using said solvents results in high encapsulation concentration of protein in GML/stabilizer matrix, and the production of small size nanoparticles. A skilled artisan will appreciate that these nanoparticles can enter the alveoli of the lungs by deep intramucosal delivery.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Example 1

CoV or nCoV-2019 antibodies will be used to prevent and CoV related diseases and/or Covid-2019, respectively in a variety clinico-epidemiological settings.

The dose of drug required is determined by the severity of the risk of developing CoV related diseases or Covid-2019 and the body weight of the individual. Prophylactic administration is given via the intramuscular route; intravenous administration is given in therapeutic applications in subjects who have already had symptoms attributable to CoV related diseases or Covid-2019 and where large doses of drug and a rapid effect are sought.

CoV or nCoV-2019 antibodies are administered prophylactically to individuals who have been exposed to CoV or nCoV-2019, respectively. These include all individuals in or travelling to an pendemic area, individuals who have been exposed to actually infected or suspected infected animals, individuals who have been exposed to subjects ill with the CoV related diseases or Covid-2019 and to individuals whose occupation puts them in contact with infected animals or humans. These individuals get the antibodies by the intramuscular route (IM), although intravenous administration is also acceptable. Individuals who are ill with CoV related diseases or Covid-2019, or suspected of being so, receive therapeutic doses of antibodies which are likely to be greater than prophylactic doses.

### Example 2

### Isolation and Manufacture

CoV or nCoV-2019 antibodies are manufactured from human plasma collected by automated plasmapheresis, and is termed source plasma or hyperimmune source plasma. In this procedure, the donor is connected to a special plasmapheresis machine for approximately 45 minutes, which automatically removes whole blood from the donor, separates the cellular elements from the liquid plasma, returns the cellular elements to the donor while retaining the plasma.

Suitable healthy donors are ascertained by a standard donor health screening questionnaire; by screening their sera or plasma for the presence of antibodies to CoV or nCoV-2019. and by measurement of the titer or quantity of antibodies present. Antibodies are acquired by two methods: first, through natural exposure to CoV or nCoV-2019 (with our without overt symptoms) or second, by deliberate immunization with attenuated Avian CoV or nCoV-2019, antigenic fragments thereof or their combinations. In certain cases, an immune system booster shal be co-administered as well.

Individuals who do not have detectable antibody in their plasma/serum are offered to receive active immunization to CoV or nCoV-2019 (CoV or nCoV-2019 vaccine). After immunization, their antibody levels are measured, and once suitable antibody titers are developed, these individuals undergo plasmapheresis in quantities and frequencies according to local protocols and regulations. This includes collecting about 800-850 mL of plasma per procedure two times per week. Immediately after collection, the plasma is frozen and stored at no more than −18° C. until further processing and purification. All collected plasma is tested for all the appropriate communicable disease markers as required by regulatory agencies.

Cohn Fractionation. Cohn plasma fractionation is used for the manufacture of a variety of plasma derivatives including a variety of normal immunoglobulin preparations (e.g. Immune Serum Globulin, Intravenous immune Globulin), immune globulin preparations (e.g. Rabies Immune Globulin, Rh Immune globulin and many others) as well as other purified proteins such as Albumin (Human), anti-hemophilic factor (factor VIII) and others.

For the manufacture of the antibodies for CoV or nCoV-2019, Cohn fractions II+III are generated by alcohol precipitation and are then further purified yielding an immunoglobulin product with an IgG content of greater than 90%. The final product is formulated at an appropriate pH—at or near 7.0-7.4 for the I.M. preparation; lower pH for the I.V. preparation and adjusted to the appropriate titer. Stabilizers may be added to improve shelf life. The product is presented in solution, but lyophilization might be used as well.

Preparatory Chromatography. In preparatory chromatography, either ion exchange chromatography or affinity chromatography or a combination of the two are used. Ion exchange chromatography is used for the manufacture of various hyperimmune globulin products such as Rabies Immune Globulin or Rh Immune Globulin.

The final product using chromatographic methods has an IgG content of greater than 90%. The final product is formulated at an appropriate pH at or near 7.0-7.4 for the I.M. preparation; lower for the I.V. preparation and adjusted to the appropriate titer. Stabilizers are added to improve shelf life. The product is presented in solution, or in a lyophilized form.

### Example 3

### Formulations, Compound Preparation and Efficacy Tests

Multiple testing methods are applied to screen the potency and characteristics of the disclosed compositions. To test the efficacy of the anti SARS-COV-2 Ig, Coronavirus strains from the CDC recommended list is expanded in Biosafety 4 cabinets in cultures. Specifically, human 229E (alpha coronavirus) and OC43 (beta coronavirus) from the ATCC, are expanded in Vero Cells (ATCC) in chemically defined NutriVero XF medium (Biological Industries Kibbutz Beit Haemek Ltd.). In a different experimental condition, culture of SARS-CoV-2 (2019-nCoV) will be cultured. Functional synergy and/or enhancement screened in culture by viral cytotoxicity method. The PDA method is used to determine correct dose and formulation efficacy.

Particle engineering approach is applied to design transmucosal and intranasal formulations to solve challenges of intrapulmonary delivery. Ig and/or a ribonuclease are encapsulated in Bioxomes nanoparticles. Bioxosomes are prepared from cell membranes by mild solvent isolation methods, followed by extrusion and/or ultrasonication. This method results in ˜250 nm sized Bioxomes. Such particles may enter bronchial deep tracheal site in intralung delivery, and can penetrate intracellularly, which is essential to catch viral infection that entrapped in host cells.

In a further experimental condition, nanoemulsion of Immunomer of Ig and/or a ribonuclease is prepared as a sterile spray. This liquid spray formulation composed of 1-10 mg ribonuclease/Ig is dispersed in 1-3 mg/ml of GML to reach its critical micelles concentrations to prepare Immunomers in the presence of trehalose/HSA stabilizers an aqueous base. To improve formulation for intrapulmonary delivery, said Immunomers are formulated to form nanospheres at ionization radiation technology via intramolecular crosslinking by gamma irradiation of globular protein. The size of these nanospheres for transmucosal delivery result in nanospheres of <100 nm, dispersed in colloidal dispersion. Selected gamma irradiation dose (5-20 kGy at the rate of more than 1 kGy per hour) result in high encapsulation concentration of protein in GML/stabilizer mixture matrix with the desired particles smaller than 100 nm that have capacity to enter alveoli of the lungs for deep intramucosal delivery. Small scale production of product prototypes in gram qualities for preclinical testing and further scale up to clinical trials is carried. To specify batch release, measurable parameters of particle engineering such as entrapment capacity of nano-vehicles is recorded. The physicochemical properties of nano-vehicles are studied for determining particle size and distribution parameters, agglomeration, concentration, dissolution and release of active compound.

### Example 4

### Bioxome Development

Corona Virus will be expanded in Vero Cells in chemically defined NutriVero medium. Synergistic methods will be screened ex vivo by viral cytotoxicity functional method. At this stage 3 types of bioconvergence approaches will be applied, utilizing synergistic anti-viral and particle engineering to result in nanocarriers to focus on specific challenges of delivery to respiratory system—most vulnerable for viral attack. Approach will be designed as acute treatment and also a preventive vaccine.

Bioxomes will be prepared from cell membranes by mild solvent isolation methods, followed by extrusion or/and ultrasonication method to encapsulate Ranpirnase. This method results in ˜250 nm sized Bioxomes. Such particles may enter bronchial deep tracheal site of intra-lung delivery and intracellular penetration that is of essence to reach viral infection that entrapped in host cells.

Particle Engineering

Small scale production of product prototypes (10-20) will be prepared and measurable parameter of particle engineering such as the entrapment capacity of nano-vehicles will be tested with the help of fluorescent dye. Fluorescent dumb/dye molecules will be introduced to the nano-vehicle and then fluorescent emission and excitation will be recorded.

Physicochemical Characterization

The nano-vehicles will be studied for following parameters. The range of product specification will be included into QC specifications and COA design:
a. Solubility
b. Particle size and distribution parameters
c. Agglomeration
d. Concentration
e. Dissolution and release

Stability and Shelf-Life Testing

The stability and shelf-life of the nanocarriers will be tested against storage conditions, temperature, humidity and light. Stress testing of the nano-vehicles will be tested at different temp, pH and humidity conditions at dark package and at artificial daylight lamp.

### Example 5

### Recombinant Ranpirnase (Ranp) Production

In the below example, the signal peptide region of Ranpirnase (Ranp) is inferred according to the known cleavage sites in other RNases. The mature peptide region of the Ranp gene is subcloned into the bacterial expression vector pFLAG CTS (Kodak, New Haven, Conn.) and verified by sequencing. Optionally, a synthetic gene for Ranp with bacterial preferential codons and the selected vector, e.g. pET11c can be used.

Induction of the T7 lac promoter leads to the expression of intracellular protein in high yield that aggregates and accumulates in inclusion bodies. A yield of around 100 mg of protein in inclusion bodies per liter of medium is obtained in TB medium, and expression in minimal medium M9 yielded around 30 mg of protein per liter of medium. Ranp represents more than 70% of the total protein in inclusion bodies after 2-5 h of culture following IPTG induction.

Construction of the Ranp expression plasmid is performed using the plasmid construct pFLAG CTS/Ranp. Plasmid pFLAG CTS/Ranp is subjected to polymerase chain reaction for subcloning of Ranp gene into the pET11c plasmid vector. The final sequence corresponds to Ranp lacking the leader sequence and having an additional functional block, e.g. N-terminal methionine residue (pET11c/Ranp). Longer incubation leads to the accumulation of other cellular proteins and, consequently, a lower yield in the refolding step.

Refolding of recombinant protein is achieved by rapid dilution of denatured reduced protein in renaturing buffer. The best yield is obtained when the GSH/GSSG ratio is 4:1, and the protein is added to the refolding buffer at a final concentration of 50 mg/ml.

Protein expression in the E. coli BL21(DE3) strain (Novagen, Madison, Wis.), folding of the proteins from inclusion bodies. and the purification steps are carried out as described below. Protein concentration could rise up to 200 mg/ml if added stepwise, allowing 1 h incubation between each loading. The renatured protein is easily purified to homogeneity by cation exchange chromatography with a final yield of 5-10 mg of purified protein per 1 liter of culture. The purified recombinant protein is analyzed by 15% SDS-PAGE stained with Coomassie Blue or by SDS-PAGE containing either poly(C) or poly(U) as substrates for activity staining. Analysis of the E. coli BL21(DE3) expression strain without the expression plasmid by means of SDS-PAGE activity staining indicates that there is no other protein with RNase activity in the insoluble intracellular fraction.

### Example 6

### Ribonuclease Activity

The ribonuclease activity of the recombinant proteins against a standard yeast tRNA substrate is measured in 40 mM sodium phosphate buffer (pH 7.4) at 25° C. Purified RNase is added into 0.8 ml of the aforementioned buffer with 1.42 nmol tRNA. The reaction is stopped by 0.5 ml of 20 mM lanthanum nitrate with 3% perchloric acid, and insoluble tRNA is removed by centrifugation. The amount of solubilized tRNA is determined by ultraviolet absorbance at 260 nm.

The catalytic activity of the Ranp is determined as the pmol of RNA digested per Ranp 7. For comparison, we also are examining the activities of selected natural purified standards of ranpirnase purified from frog oocytes at >99% purity using 1 pmol natural enzyme standard. The activity of recombinant Ranp in reducing the infectivity of respiratory syncytial virus (RSV) on HEp-2 human epithelial cells is examined by the quantitative shell vial amplification technique: recombinant Ranp is added to Hep-2 monolayers growing on coverslips (50 000 cells/coverslip) followed by ˜2000 plaque-forming units (infectious units) of RSV-B [American Type Culture Collection (ATCC), Manassas, Va.].

The vials containing virus and target cells are centrifuged for 60 min at 500 g at room temperature and then incubated at 37° C. overnight, after which the coverslips are washed, acetone fixed, and stained with FITC-anti-RSV with methylene blue counterstain (Chemicon, Temecula, Calif.).

Infected cells are identified by fluorescence microscopy. Ranp is highly cationic, therefore has bifunctional activity cytotoxic (most probably due high cationic interaction with negative moieties) and enzymatic RNAse effect on viral double strand RNA.

For labeling assay Ranp is labelled with the Alexa Fluor 488 fluorophore, following the manufacturer's instructions, as previously described: to 0.5 mL of a 2 mg/mL protein solution in phosphate saline buffer (PBS), 50 μL of 1 M sodium bicarbonate, pH 8.3, are added. The protein is incubated for 1 h at room temperature, with the reactive dye, with stirring, following the manufacturer's conditions. The labelled protein is separated from the free dye by a PD10-desalting column.

### Example 7

### Ranpirnase Encapsulation into Bioxome™

Fluorescently-labeled (as above) Ranp is encapsulated into Bioxomes prepared co-extracted with glycerol monolaurate (GML) prepare GML-bioxomes with higher efficiency of encapsulation than into pure Bioxomes.

Bioxomes are prepared from adipose tissue-derived stem cells (Mesenchymal Stromal Cells, MSCs), as described in WO2019198068 (incorporated herein by reference). Encapsulation is performed as described below.

Cellular membrane lipids are extracted from 5×10\hat{ }8 MSCs by adding 4.5 ml Hexane:Isopropanol (HIP) solution (1:1, Volume/Volume) to the cell pellet and resuspending the resulting solution. Upon vortexing, the HIP/Cell suspension is centrifuged at 20,000 G, 4° C. for 30 min. Supernatant is transferred to the clean tube (keeping ˜0.5 ml of the suprnatant above the pellet, in order not to contaminate the supernatant). The collected supernatant is further lyophilized for 1-3 days, or until the formation of dry lipid film.

Working Ranpirnase solution is prepared by dissolving 4.5 mg of Ranp powder in 4.5 ml saline (to achieve lmg/ml solution) and kept at 4° C. until use. Lipid (Bioxome)/Ranp suspension is prepared under sterile conditions as described below. All samples are pre-chilled and kept at 4° C. throughout the procedure. The lyophilized lipid film is re-suspended in 4.5 ml of 1 mg/ml Ranp solution. Two hundred (200) microL of the samples are sent to HPLC analysis of the HIP traces remnants.

Crude Bioxome/Ranpirnase solution is prepared by sonication of lipids/Ranpirnase solution on cold block (pre-chilled to 4°, with the ultrasonicator set to 40% intencity and in 3 sonication pulses of 6 seconds duration each), with 24 seconds resting between the pulses. After the sonication, 10 microL aliquot is removed for measuring the particles (micelles) size and concentration, by NanoSight.

The free (non-encapsulated) Ranp is washed by loading the Bioxome/Ranp crude suspension on Amicon Ultra filter and centrifuging the filter at 4000 g for 15 min at 4° C., or until the volume is reduced to <0.5 ml. Upon emptying the lower chamber, the filtration chamber is washed three times with 15 of fresh sterile saline, followed by centrifugation. The resulting Bioxome-encapsulated Ranpirnase is recovered to a clean tube and diluted to achieve the volume of 4.5 ml and analyzed as follows: (1) Particle size and concentration are measured by Nanosight: (2) the encapsulation yield is measured by Running 20 μl of the Bioxome-encapsulated Ranpirnase on 20-4% SDS-PAGE along with calibration Ranpirnase standards and calculating the Encapsulation Yield as μg Ranpirnase/10\hat{ }6 Bioxome particles.

When GML-Bioxomes are used to encapsulate Ranp, the loading of Ranp is significantly higher as compared with the loading in Bioxomes without GLPand. In addition, the release of Ranp is prolonged significantly. The cytotoxicity of the Ranp-loaded GML-Bioxomes is much higher than that of free Ranp because of the endocytic cellular uptake of the particles. Strong targeting in vivo to lungs and liver has advantage to target respiratory and hepatic virus infections.

### Example 8

### Spray Development

Glycerol monolaurate (GML) is a natural surfactant permeability enhancer method for transmucosal delivery of a ribonuclease or Ranpirnase to enhance its anti-viral bioactivity. In this method, the GML will be used at its critical micelles concentrations to prepare the therapy in the presence of carbohydrate/HSA stabilizers an aqueous base. The size of these nanospheres for transmucosal delivery should be <100 nm, which is the optimal size for colloidal dispersion.

Optionally, protein compatible solvent will be used (similar to the Bioxome method) with an optional gamma irradiation dose (5-20 kGy at the rate of more than 1 kGy per hour) that results in high encapsulation concentration of protein in GML/stabilizer matrix and producing small size nanoparticles. These nanoparticles will have capacity to enter the alveoli of the lungs via deep intramucosal delivery.

### Example 9

### Pre-Clinical Evaluation

Murine SARS-CoV virus infection in-vivo model establishment, ribonuclease or Ranpirnase Potency and POC as monotherapy and as combination with Bioxomes—Intravenous or Intranasal route

BALB/c male and/or female mice (6-8 weeks of age) will be allowed to habituate for 1 week prior to the study. The animals will be housed in climate-controlled quarters (24° C. at 50% humidity) with 12 h light/dark cycles and had free access to food and water. All experimental protocols will be approved by an Institutional Animal Welfare Committee.

A. Model establishment of Severe acute respiratory syndrome (SARS)-CoV infection.

Group allocation:
a. Baseline group (without infection) (n=10)
b. 1×102 PFU (n=10)
c. 1×103 PFU (n=10)
d. 1×105 PFU (n=10)

B. Ranpirnase potency (0.1-5 μg/ml) as monotherapy (via IV or IN)

Group allocation:
a. Baseline group (without infection) (n=10)
b. Negative control group (infected, no active therapy) (n=10)
c. Ranpirnase low dose IV administration (n=10)
d. Ranpirnase medium dose IV administration (n=10)
e. Ranpirnase high dose IV administration (n=10)
f. Ranpirnase low dose IN administration (n=10)
g. Ranpirnase medium dose IN administration (n=10)
h. Ranpirnase high dose IN administration (n=10)

C. Combination Ranpirnase and Bioxomes—route of administration according to B.
a. Baseline group (without infection) (n=10)
b. Negative control group (infected, no active therapy) (n=10)
c. Only Ranpirnase*IV/IN administration (n=10)
d. Only Bioxomes (high dose)
e. Ranpirnase with bioxomes ratio A IV/IN administration (n=10)
f. Ranpirnase with bioxomes ratio B IV/IN administration (n=10)
g. Ranpirnase with bioxomes ratio C IV/IN administration (n=10)

*Ranpirnase dose TBD according to prior study.

D. Proof of concept
a. Baseline group (without infection) (n=10)
b. Negative control group (infected, no active therapy) (n=10)
c. Ranpirnase optimal dose IV/IN administration (from step B) (n=20)
d. Ranpirnase & Bioxomes (optimal ratio from step C) IV/IN administration (n=20)

SARS-Cov Model induction: Murine or human strain suitable to work up to BSL-2 laboratories. Animals will be anesthetized with a mixture of ketamine-xylazine and infected intranasally either with the virus or with phosphate-buffered saline (PBS) in a dose of 50 μl. Virus doses ranged from 1×102 to 1×105 PFU/50-μl dose, diluted in PBS prior to intranasal administration (Day C W et al. Virology 2009).

Study duration: 4 weeks (one-week STZ, 4 weeks treatment)

Handling: 5 weeks (including one-week acclimation)

Animals: BALB/c male and/or female mice (6-8 weeks of age)

Examinations

Body weight: 3 times a week.

Morbidity & mortality check: Daily.

Clinical observation: 3 times a week.

Termination

Animals will be sacrificed four weeks following treatment initiation, gross pathology will be performed, and lungs tissues will be collected.

Lung samples from each test group will be pooled and homogenized in MEM solution and assayed in duplicate for infectious for virus yield assays using triplicate wells of Vero 76 cells.

Lung tissues will be fixed in PBS-4% paraformaldehyde (pH 7.3); tissues will be embedded in paraffin; and 5-μm-thick sections will be prepared for histopathology. Extent of inflammation will be determined, sections will be stained with hematoxylin and eosin (H&E) and will be scored from 1 to 5 for overall inflammation, eosinophilia, neutrophilia, alveolitis, bronchiolar denudation, and edema.

Hyaline membrane formation will be evaluated in lungs in comparison to control groups.

Blood will be harvested from all animals (by terminal bleeding), serum separation and complete blood count and biochemistry panel will be performed.

Multiplex ELISA will be performed at different time (serum and lung samples) points following challenge and treatment to measure IL-1α and IL-6, and chemokines MIP-1α, MCP-1, and RANTES.

### Example 10

### Ranpirnase Efficacy In Vitro

The objective of this experiment was to compare the efficacy of ranpirnase and FDA approved drugs for reducing SARS-CoV-2 concentration in vitro.

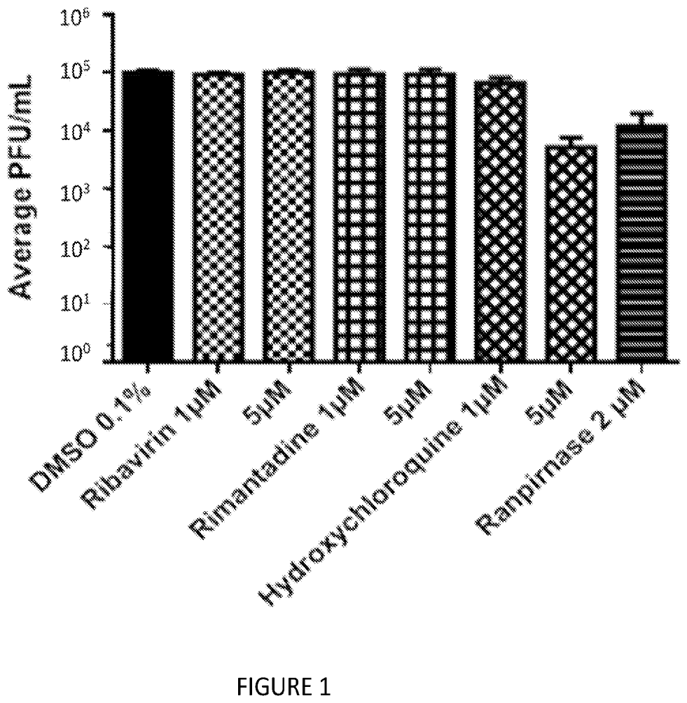

Ranpirnase was one of 2 drugs to reduce virus concentrations in the assay (FIG. 1) Similarly, UTHSC tested ranpirnase in their high-throughput screening (HTS) method for SARS-CoV-2 with the 50% effective dose (EC50)˜6 μM.

Based on the in vitro activity against SARS-CoV-2, ranpirnase is expected to be active against SARS-CoV-2 in a hamster infection study, in which hamsters will be challenged with SARS-CoV-2 with analysis of lung infection on days 3, 5, and 7 to evaluate virus reduction in these tissues.

### Example 11

### Clinical Study of Ranpirnase for Treating Covid-19

The proposed clinical study is expected to present minimal risk to participants while contributing to development of a safe, efficacious COVID-19 therapeutic. The clinical research will be conducted in accordance with applicable International Conference on Harmonisation of Technical Requirements for Registration of Pharmaceuticals for Human Use (ICH) guidelines and FDA regulations and guidance. Ranpirnase will be tested in a human Phase II/III clinical trial to evaluate the technical feasibility of ranpirnase to reduce viral load and risk of an IL-6 cytokine storm in COVID-19 patients. Table 1 shows the Phase II/III protocol synopsis we are proposing to conduct. The study is designed for participation by adult (≥21 years old) COVID-19 patients. The clinical study will enroll up to 45 subjects with a 3:1 treatment:placebo (standard of care) ratio. All inclusion and exclusion criteria must be met for eligibility. Evaluation of this investigational therapeutic will include laboratory tests, medical history, physical assessment by clinicians, and subject self-assessment (if appropriate). The hypotheses are that ranpirnase is safe and reduces the viral burden in COVID-19 patients. Primary endpoints will be safety and efficacy. The assessment of product safety will include clinical observation and monitoring of hematological, and chemical parameters while efficacy assessment will evaluate virological measures.

TABLE 1
Protocol Synopsis for Phase II/III Clinical Study

| Title | A Phase II/III randomized, double-blind, placebo-controlled, | | |
|---|---|---|---|
| | multiple-ascending-dose study to evaluate the safety, efficacy, | | |
| | and tolerability of ranpirnase in COVID-19 patients | | |
| Objectives/Endpoints | Safety, Efficacy, Tolerability/Safety and Efficacy | | |
| Number of | 1-3 sites/45 Subjects | | |
| Sites/Subjects | | | |
| Study & Participant | 6 months for Study/1 month for participants | | |
| Duration | | | |
| Study Design | Subjects will be randomized in a 3:1 ratio to receive 1 | | |
| | intravenous (IV) injection of the study product or placebo per | | |
| | day for 5 days. Equal distribution of male and female. One (1) | | |
| | week between groups for evaluation of safety data. | | |
| | Regimen | Dose* | # of Subjects |
| | Group 1 | 0.0081 mg/kg | 9 |
| | Group 2 | 0.016 mg/kg | 9 |
| | Group 3 | 0.032 mg/kg | 9 |
| | Group 4 | 0.065 mg/kg | 9 |
| | Group 5 | 0.10 mg/kg | 9 |

| | *Dosing subject to change based on nonclinical data. |
|---|---|
| Safety Procedures | Adverse event assessments, physical examinations, and |
| | laboratory assessments |
| Variables for | Spontaneous and solicited AEs; SAEs |
| Evaluation | |

Having described preferred embodiments of the invention, it is to be understood that the invention is not limited to the precise embodiments, and that various changes and modifications may be effected therein by those skilled in the art without departing from the scope or spirit of the invention as defined in the appended claims.

1. A composition comprising a ribonuclease and a bioxome, an exosome or a combination thereof.

2. The composition of claim 1, wherein said ribonuclease is selected from a group comprising RNase A, RNase H, RNase III, RNase L, RNase P, RNase PhyM, RNase T1, RNase T2, RNase U2, RNase V, PNPase, RNase PH, RNase R, RNase D, RNase T, oligoribonuclease, exoribonuclease I, exoribonuclease II, binase, MCPIP1, eosinophil cationic protein (ECP), eosinophil derived neurotoxin (EDN), RNase 3, ranpirnase, rAmphinase, rAmphinase 2, bovine seminal RNase (BS_RNase).

3. The composition of claim 1, wherein said ribonuclease comprises ranpirnase.

4. The composition of claim 1, for use in treating a viral disease.

5. The composition of claim 4, wherein said viral disease is caused by a virus selected from a group comprising severe acute respiratory syndrome coronavirus 2 (SARS-CoV-2), an adenovirus, a herpesvirus, a papillomavirus, a polyomavirus, a poxvirus, an hepadnavirus, a parvovirus, an astrovirus, a calicivirus, a picornavirus, a coronavirus, a flavivirus, a togavirus, a hepevirus, a retrovirus, an orthomyxovirus, an arenavirus, a bunyavirus, a filovirus, a paramyxovirus, a rhabdovirus, a reovirus, Herpes simplex type 1, Herpes simplex type 2, Varicella-zoster virus, Epstein-Barr virus, Human cytomegalovirus, human herpesvirus type 8, human papillomavirus, BK virus, JC virus, smallpox, Hepatitis B virus, parvovirus B19, human astrovirus, Norwalk virus, coxsackievirus, hepatitis A virus, poliovirus, rhinovirus, severe acute respiratory syndrome virus, hepatitis C virus, yellow fever virus, dengue virus, West Nile virus, TBE virus, Rubella virus, Hepatitis E virus, Human immunodeficiency virus (HIV), Influenza virus, Lassa virus, Crimean-Congo hemorrhagic fever virus, Hantaan virus, Ebola virus, Marburg virus, Measles virus, Mumps virus, Parainfluenza virus, Respiratory syncytial virus, Rabies virus, Hepatitis D, Rotavirus, Orbivirus, Coltivirus, Banna virus, or any combination thereof.

6. The composition of claim 4, wherein said viral disease is selected from a group comprising acute hepatitis, AIDS, aseptic meningitis, bronchiolitis, Burkitt's lymphoma, chickenpox, chronic hepatitis, common cold, congenital rubella, congenital varicella syndrome, congenital seizures in the newborn, croup, cystitis, cytomegalic inclusion disease, fatal encephalitis, gastroenteritis, German measles, gingivostomatitis, hepatic cirrhosis, hepatocellular carcinoma, herpes labialis, cold sores, herpes zoster, Hodgkin's lymphoma, hyperplastic epithelial lesions, warts, laryngeal papillomas, epidermodysplasia verruciformis, infectious mononucleosis, influenza, influenza-like syndrome, Kaposi sarcoma, keratoconjunctivitis, liver, lung and spleen diseases in the newborn, malignancies, cervical carcinoma, squamous cell carcinomas, measles, multicentric Castleman disease, mumps, myocarditis, nasopharyngeal carcinoma, pericarditis, pharyngitis, pharyngoconjunctival fever, pleurodynia, pneumonia, poliomyelitis, postinfectious encephalomyelitis, premature delivery, primary effusion lymphoma, rabies, Reye syndrome, severe bronchiolitis with pneumonia, skin vesicles, mucosal ulcers, tonsillitis, pharyngitis, or combination thereof.

7. The composition of claim 4, wherein said viral disease comprises Covid-19, or wherein said viral disease is caused by severe acute respiratory syndrome coronavirus 2 (SARS-CoV-2).

8. The composition of claim 1, further comprising immunoglobulins, fragments thereof, antibodies, or combinations thereof, obtained from a plasma of a subject immune to said viral disease.

9. The composition of claim 1, further comprising immune cells.

10. A pharmaceutical composition comprising the composition of claim 1 and an excipient.

11. The pharmaceutical composition of claim 10, wherein said pharmaceutical composition is formulated for intravenous, oral, intranasal, pulmonary, transdermal, parenteral, intraperitoneal, intracranial, intramuscular, subcutaneous, intratracheal, or transmucosal delivery, or any combination thereof.

12. A method for treating a viral disease in a subject in need thereof, the method comprising administering a composition comprising a ribonuclease and a bioxome, an exosome or a combination thereof.

13. The method of claim 12, wherein said ribonuclease is selected from a group comprising RNase A, RNase H, RNase III, RNase L, RNase P, RNase PhyM, RNase T1, RNase T2, RNase U2, RNase V, PNPase, RNase PH, RNase R, RNase D, RNase T, oligoribonuclease, exoribonuclease I, exoribonuclease II, binase, MCPIP1, eosinophil cationic protein (ECP), eosinophil derived neurotoxin (EDN), RNase 3, ranpirnase, amphinase, rAmphinase 2, bovine seminal RNase (BS_RNase).

14. The method of claim 12, wherein said ribonuclease comprises ranpirnase.

15. The method of claim 12, wherein said viral disease is caused by a virus selected from a group comprising severe acute respiratory syndrome coronavirus 2 (SARS-CoV-2), an adenovirus, a herpesvirus, a papillomavirus, a polyomavirus, a poxvirus, an hepadnavirus, a parvovirus, an astrovirus, a calicivirus, a picornavirus, a coronavirus, a flavivirus, a togavirus, a hepevirus, a retrovirus, an orthomyxovirus, an arenavirus, a bunyavirus, a filovirus, a paramyxovirus, a rhabdovirus, a reovirus, Herpes simplex type 1, Herpes simplex type 2, Varicella-zoster virus, Epstein-Barr virus, Human cytomegalovirus, human herpesvirus type 8, human papillomavirus, BK virus, JC virus, smallpox, Hepatitis B virus, parvovirus B19, human astrovirus, Norwalk virus, coxsackievirus, hepatitis A virus, poliovirus, rhinovirus, severe acute respiratory syndrome virus, hepatitis C virus, yellow fever virus, dengue virus, West Nile virus, TBE virus, Rubella virus, Hepatitis E virus, Human immunodeficiency virus (HIV), Influenza virus, Lassa virus, Crimean-Congo hemorrhagic fever virus, Hantaan virus, Ebola virus, Marburg virus, Measles virus, Mumps virus, Parainfluenza virus, Respiratory syncytial virus, Rabies virus, Hepatitis D, Rotavirus, Orbivirus, Coltivirus, Banna virus, or any combination thereof.

16. The method of claim 12, wherein said viral disease is selected from a group comprising acute hepatitis, AIDS, aseptic meningitis, bronchiolitis, Burkitt's lymphoma, chickenpox, chronic hepatitis, common cold, congenital rubella, congenital varicella syndrome, congenital seizures in the newborn, croup, cystitis, cytomegalic inclusion disease, fatal encephalitis, gastroenteritis, German measles, gingivostomatitis, hepatic cirrhosis, hepatocellular carcinoma, herpes labialis, cold sores, herpes zoster, Hodgkin's lymphoma, hyperplastic epithelial lesions, warts, laryngeal papillomas, epidermodysplasia verruciformis, infectious mononucleosis, influenza, influenza-like syndrome, Kaposi sarcoma, keratoconjunctivitis, liver, lung and spleen diseases in the newborn, malignancies, cervical carcinoma, squamous cell carcinomas, measles, multicentric Castleman disease, mumps, myocarditis, nasopharyngeal carcinoma, pericarditis, pharyngitis, pharyngoconjunctival fever, pleurodynia, pneumonia, poliomyelitis, postinfectious encephalomyelitis, premature delivery, primary effusion lymphoma, rabies, Reye syndrome, severe bronchiolitis with pneumonia, skin vesicles, mucosal ulcers, tonsillitis, pharyngitis, or combination thereof.

17. The method of claim 12, wherein said viral disease comprises Covid-19, or wherein said viral disease is caused by severe acute respiratory syndrome coronavirus 2 (SARS-CoV-2).

18. The method of claim 12, wherein said composition is administered by intravenous, oral, intranasal, pulmonary, transdermal, parenteral, intraperitoneal, intracranial, intramuscular, subcutaneous, intratracheal, or transmucosal route, or any combination thereof.

19. (canceled)

20. (canceled)

21. A method for treating or preventing a viral disease in a subject, comprising administering a composition, wherein said composition comprises a ribonuclease and wherein said composition is loaded into a bioxome, an exosome, or a combination thereof.

22. A method for treating or preventing a viral disease in a subject, comprising administering a composition, wherein said composition comprises a ribonuclease and wherein said composition is administered with a bioxome, an exosome, or a combination thereof.
